(19) United States
(12) Patent Application Publication
(10) Pub. No.: US 2023/0414619 A1
(43) Pub. Date: Dec. 28, 2023

(54) G9A INHIBITORS AND EZH2 INHIBITORS AS MULTIFACETED COVID-19 THERAPEUTICS

(71) Applicant: The University of North Carolina at Chapel Hill, Chapel Hill, NC (US)

(72) Inventor: Xian Chen, Chapel Hill, NC (US)

(21) Appl. No.: 18/035,831

(22) PCT Filed: Nov. 15, 2021
(86) PCT No.: PCT/US2021/059350

§ 371 Date: May 8, 2023

Related U.S. Application Data
(60) Provisional application No. 63/113,211, filed on Nov. 13, 2020.

(51) Int. Cl. A61K 31/517; A61K 31/496; G01N 33/50; A61P 31/14

(52) U.S. Cl. CPC A61K 31/517; A61K 31/496; G01N 33/5023; A61P 31/14

(57) ABSTRACT

Method of treating subjects suffering from symptoms related to a coronavirus infection, including COVID-19 viral infections, are provided. The methods include administering to a subject an inhibitor of G9a, an inhibitor of Ezh2, or combinations of the two. Such inhibitors can be small molecule inhibitors, including for example UNC0642 and UNCI 999. Methods of blocking G9a translational regulation of inflammation in a subject are also provided. Additionally, methods of identifying a compound to treat and/or prevent chronic and/or acute inflammation in a subject, based on identifying compound that block G9a translational regulation, are provided.

### RELATED APPLICATIONS

The presently disclosed subject matter claims the benefit of U.S. Provisional Patent Application Ser. No. 63/113,211, filed Nov. 13, 2020, the disclosure of which is incorporated herein by reference in its entirety.

### STATEMENT OF GOVERNMENT INTEREST

This invention was made with government support under Grant Number GM133107 awarded by the National Institutes of Health. The government has certain rights in the invention.

### REFERENCE TO SEQUENCE LISTING

The Sequence Listing associated with the instant disclosure has been electronically submitted to the United States Patent and Trademark Office as a 2 kilobyte ASCII text file created on Nov. 11, 2021, and entitled “Sequence Listing 421-512-PCT ST25.txt”. The Sequence Listing submitted via EFS-Web is hereby incorporated by reference in its entirety

### TECHNICAL FIELD

Provided herein are G9a inhibitors and Ezh2 inhibitors as multifaceted COVID-19 therapeutics. Also provided herein are methods of treating SARS-CoV-2 infected subjects.

### BACKGROUND

The Coronavirus Disease (COVID-19) pandemic, caused by severe acute respiratory syndrome coronavirus-2 (SARS-CoV-2), is an unprecedented global public health crisis. High mortality is observed for SARS-CoV-2-infected patients who have pre-existing chronic conditions such as recovery from sepsis, chronic pulmonary diseases, metabolic diseases (e.g., diabetes), asthma, cardiovascular diseases, thrombosis, chronic liver disease and cirrhosis, and cancer. A dysregulated immune system or hyperinflammatory response to SARS-CoV-2 infection is the leading cause of severe illness and mortality. Correspondingly, excessive release of inflammatory factors in a ‘cytokine storm’ aggravates acute respiratory distress syndrome (ARDS) or widespread tissue damage, which results in respiratory or multi-organ failure and death. The immunopathologic characteristics of COVID-19 include reduction and functional exhaustion of T cells (lymphopenia) and increased levels of serum cytokines (hyperinflammation). Ordinarily, macrophages regulate the innate immune response to viral threats by producing inflammatory molecules that activate T cells for viral containment and clearance. However, macrophage function/activation is dysregulated in severe COVID-19 patients, and proinflammatory monocyte-derived macrophages are abundant in their bronchoalveolar lavage fluids. These observations indicated the crucial effects of dysregulated macrophages in SARS-CoV-2 immunopathogenesis and associated cytokine storms. Therapeutic options have been severely restrained by a lack of understanding of the pathways and mechanisms that trigger the SARS-CoV-2-induced hyperinflammatory response.

What is needed is a better understanding of the pathways and mechanisms that trigger the SARS-CoV-2-induced hyperinflammatory response, and host pathways aberrantly activated in patient macrophages. Moreover, what is needed are new therapeutics, therapeutic methods and treatments.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

Provided in some embodiments are methods of treating a subject suffering from symptoms related to a coronavirus infection and/or COVID-19, the method comprising administering to the subject a therapeutically effective amount of an inhibitor of G9a, an inhibitor of Ezh2, and/or combinations thereof. In some embodiments the subject is suffering from COVID-19. In some embodiments, the subject is suffering from SARS-CoV-2 pathologic pathways related to a host response and viral replication from the coronavirus infection and/or COVID-19. In some aspects, the subject is suffering from a hyperinflammatory response mediated by SARS-CoV-2-dysregulated macrophage activation resulting in a cytokine storm.

In some embodiments, the G9a inhibitor comprises a small molecule capable of reducing and/or inhibiting translational regulatory processes associated with G9a. In some aspects, the G9a inhibitor comprises UNC0642, and the EZH2 inhibitor comprises UNC1999.

In some aspects, the subject is co-administered both the inhibitor of G9a and the inhibitor of Ezh2. In some aspects, the administration of the inhibitor of G9a and/or the inhibitor of Ezh2 reduces and/or inhibits coronavirus replication in the subject. The administration of the inhibitor of G9a and/or the inhibitor of Ezh2 can in some embodiments restore T cell function to overcome lymphopenia, mitigates hyperinflammation, and/or suppresses of viral replication in the subject. The administration of the inhibitor of G9a and/or the inhibitor of Ezh2 suppresses a systemic hyperinflammatory response in the subject by simultaneously inhibiting multiple components of a COVID-19 cytokine storm, wherein the components of the COVID-19 cytokine storm that are inhibited are ARDS-related proteins and/or sepsis-related proteins, optionally wherein the ARDS-related proteins and/or sepsis-related proteins are selected from the group consisting of SPP1, CCL2, IL1RN, CXCL2, SQSTM1, ANPEP, PLAU, PELI1, PROCR, DST, and FABP4.

In some embodiments, the inhibitor of G9a or Ezh2 is administered to the subject in a pharmaceutically acceptable formulation or carrier. The subject can in some aspects be a human subject.

Provided in some embodiments are methods of blocking G9a translational regulation of hyperinflammation in a subject, the method comprising administering to the subject a therapeutically effective amount of an inhibitor of G9a, wherein G9a translational regulation of inflammation in the subject is blocked or substantially reduced. The subject can in some embodiments be suffering from an infection or other condition causing chronic or acute inflammation. In some embodiments, the subject is suffering from coronavirus viral infection and/or COVID-19.

In some embodiments, the G9a inhibitor comprises a small molecule capable of reducing and/or inhibiting translational regulatory processes associated with G9a. In some embodiments, the G9a inhibitor comprises UNC0642, wherein the EZH2 inhibitor comprises UNC1999. In some embodiments, the administration of the inhibitor of G9a suppresses a systemic hyperinflammatory response in the subject by inhibiting ARDS-related proteins and/or sepsis-related proteins, optionally wherein the ARDS-related proteins and/or sepsis-related proteins are selected from the group consisting of SPP1, CCL2, IL1RN, CXCL2, SQSTM1, ANPEP, PLAU, PELI1, PROCR, DST, and FABP4. In some embodiments, the administration of the inhibitor of G9a blocks METTL3-mediated translational regulation of chronic inflammation in the subject. In some embodiments, the inhibitor of G9a is administered to the subject in a pharmaceutically acceptable formulation or carrier. In some embodiments, the subject is a human subject.

Also provided herein are methods of identifying a compound to treat and/or prevent chronic and/or hyperinflammation in a subject, the method comprising identifying a compound that blocks G9a translational regulation of inflammation in the subject. In some embodiments, the compound comprises an inhibitor of G9a, optionally a small molecule inhibitor.

Provided herein in some embodiments is the use of a composition for treating a subject suffering from symptoms related to a coronavirus infection, comprising administering to the subject the composition comprising a therapeutically effective amount of an inhibitor of G9a, an inhibitor of Ezh2, and/or combinations thereof. In some embodiments, the subject is suffering from a coronavirus infection and/or COVID-19. In some embodiments, the subject is suffering from SARS-CoV-2 pathologic pathways related to a host response and viral replication from the coronavirus infection or COVID-19. In some embodiments, the subject is suffering from a hyperinflammatory response mediated by SARS-CoV-2-dysregulated macrophage activation resulting in a cytokine storm. In some embodiments, the G9a inhibitor comprises a small molecule capable of reducing and/or inhibiting translational regulatory processes associated with G9a. In some embodiments, the G9a inhibitor comprises UNC0642, wherein the EZH2 inhibitor comprises UNC1999. In some embodiments, the subject is co-administered both the inhibitor of G9a and the inhibitor of Ezh2. In some embodiments, the administration of the inhibitor of G9a and/or the inhibitor of Ezh2 reduces and/or inhibits coronavirus replication and/or infection in the subject. In some embodiments, the administration of the inhibitor of G9a and/or the inhibitor of Ezh2 restores T cell function to overcome lymphopenia, mitigates hyperinflammation, and/or suppresses of viral replication in the subject. In some embodiments, the subject is a human subject.

These and other objects are achieved in whole or in part by the presently disclosed subject matter. Other objects and advantages of the presently disclosed subject matter will become apparent to those skilled in the art after a study of the following description, Drawings and Examples.

### BRIEF DESCRIPTION OF THE FIGURES

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A-1D. Constitutively active G9a is implicated in SARS-CoV-2 upregulated translation pathways in ET. FIG. 1A. Schematic of LFQ ChaC-MS dissection of G9a interactome in ET macrophages. FIG. 1B. Over-represented functional pathways and networks of G9a interactors in chronically inflamed macrophages (TL/ET). Physical interactions were curated from StringApp, Reactome in Cytoscape (v3.8.1). FIG. 1C. Immunoblot analysis of ET-specific associations with endogenous G9a for METTL3 and major translation regulators. The Raw264.7 macrophage lines either stably expressing shRNA for G9a knock-down (KD), or empty vector (EV) for G9a wild type. ‘EV+UNC0642’ refers to G9a inhibitor (UNC0642) treatment. (left) inputs, and (right) UNC0965 pull-down (PD). Some interactors were pulled down from the G9a KD cells because of residual G9a. FIG. 1D. Polysome analysis of G9a- or METTL3-dependent protein synthesis. Absorbance profile of sucrose density gradients showing the location of 40S and 60S ribosomal subunits, 80S monosomes, and polysomes.

FIGS. 2A-2E. G9a and METTL3 cooperate to dysregulate cell cycle and impair T cell function under ET. FIG. 2A. Enriched functional pathways overrepresented by G9a/METTL3-co-upregulated m⁶A mRNA in ET. The network was constructed by BinGO app in Cytoscape. FIG. 2B. Immunoblot of protein expression in endotoxin tolerized wild-type versus G9a knock-out (ko) or METTL3 ko THP-1 cells. FIG. 2C. LPS-induced cell cycle arrest in endotoxin tolerance condition requires G9A and METTL3. Flow cytometry analysis of the impact of G9a and METTL3 on cell cycle in different inflammatory conditions (e.g., N, NL, or TL). Cells were labeled with EdU for 0.5 h before harvesting and analyzed by flow cytometry for DNA content with DAPI and for DNA synthesis with EdU. FIG. 2D. Clonogenic survival assay of WT and KO Raw 264.7 cells in different inflammation conditions. WT cells were either treated with DMSO (0.05%) or 1 mM G9a inhibitor (UNC0642) or EZH2 inhibitor (UNC1999). * p<0.05, ** p<0.01. FIG. 2E. Depletion of METTL3 or G9a promotes the T cell activation and proliferation under the TL conditions. Histograms obtained from the CD8 T-cell activation (upper panel) and proliferation (lower panel). The proliferation and activation markers, including CD25 of P14 CD8⁺ T cells, were analyzed by flow cytometry at day 5 and day 6 after coculture with wild type, METTL3 ko or G9a ko RAW 264.7 cells that were untreated (N) or treated with an acute LPS stimulation (NL) and prolonged LPS stimulation (TL), a mimic of ET.

FIGS. 3A-3F. G9a-mediated lysine methylation is implicated in the METTL3-mediated translation in ET macrophages. FIG. 3A. (Left): G9a interacts with METTL3, the interactions were confirmed by both ‘forward’ HA-G9a IP and ‘reverse’ Flag-METTL3 IP; (middle): METTL3 specifically interacts with chronically active G9a in LPS-tolerant 293-TLR4/CD14/MD2 cells. Cell responses were monitored by p65 phosphorylation; (right): Time-course dependent interactions between HA-G9a and Flag-METTL3 under N, NL, T, or TL. FIG. 3B. Mapping the interacting domains of G9a and METTL3; (Left): Lacking the catalytic domain of G9a (CD) disrupts the interaction with METTL3; (right): C-terminus deletion (201-580) mutant of METTL3 (Flag-MS2-METTL3 1-200) abolished the interaction with G9a. FIG. 3C. G9a methylates multiple METTL3 lysine in ET. MS/MS spectra of the tryptic peptides of METTL3 bearing mono-Kme or di-Kme. b- or y-ions were labeled in blue and red, a methylated ion has a 14-dalton mass shift compared with its unmodified counterpart. FIG. 3D. LPS-induced methylation dynamics of METTL3 under different inflammatory conditions. Flag-METTL3 and HA-G9a was transiently transfected into 293-TLR4/CD14/MD2 cells which then were subjected to LPS stimulation (N, NL, T, TL) 24 h after transfection. LFQ was based on relative peak areas of the identified methylated peptides and corresponding nonmethylated counterpart. Error bars show the standard deviation from two independent experiments each with duplicates. Asterisks indicate the statistical significance: **P<0.01, *P<0.05. FIG. 3E. Removal of lysine methylation weakened METTL3 interactions with eIF3b. ‘WT’—wild type METTL3; 1(215R, K281R′— K-to-R mutant of METTL3. Interaction strength was determined by densitometry. FIG. 3F. A proposed mechanism underlying G9a methylation-activated, METTL3-mediated translation based on results disclosed herein.

FIGS. 4A-4F. Constitutively active G9a promotes translation of SARS-CoV-2-evolving pathway components. FIG. 4A. Schematic of AACT-pulse labeling translatome strategy for determining the rates of protein synthesis, degradation, and turnover in N and TL/ET conditions. Raw macrophages grown in KO/RO containing medium were pulse-labeled with K4/R6, with or without UNC0642 treatment, and harvested at 2 h. 4 h, 8 h, 24 h, 48 h, and 72 h. Decreasing and increasing K4/R6 labels were used to determine rates of protein synthesis, degradation, and turnover. FIG. 4B. Numbers of the proteins showing >2 fold-change (FC) differences in half-life upon G9a-KO or UNC0642 treatment compared with wild type cells. FIG. 4C. The wild type macrophages in ET/T had faster turnover (shorter median half-life). Distribution of protein turnover (log₂ [t₁₂]) is depicted by violin plots; sample median (horizontal line), mean (diamond shape) and IQR are shown by overlaid boxplot. Red text shows median protein turnover/half-life [in hours]. FIG. 4D. Hierarchical clustering of G9a-translated genes (mRNAs) and associated pathways. Certain G9a-translated proteins were identified as G9a/GLP interactors, non-histone substrates, or with m⁶A. Translation of numerous COVID-19 markers, SARS-CoV2 host interactors and other coronavirus-related proteins were also affected by G9a ko or inhibition in T. FIG. 4E. Protein interaction networks for G9a/METTL3 regulated m⁶A-target genes shows that G9a ko or inhibition alters protein translation dynamics for ˜59.7% of the proteins. Representative pathways from each cluster are also shown. FIG. 4F. Virus-host protein-protein interaction map depicting G9a-translated proteins identified by our translatome strategy. Human proteins are shown as circles, whereas viral proteins are represented by yellow squares. Each edge represents an interaction between a human and a SARS-CoV-2 (solid line), SARS-CoV-1 (dashed line) or MERS-CoV (dotted line) protein with several interactions shared between these three viruses. As shown, several of the G9a-translated host proteins are ET-specific G9a interactors, G9a/GLP substrates or G9a/METTL3-regulated m⁶A mRNAs. As shown, genetic perturbation of several of these G9a-translated proteins SARS-CoV-2 replication/infection with some showing the opposite effect. Pathway enrichment scores for 503 G9a-translated proteins are shown on the side. ET-specific G9a interactors [ChaC] and G9a/METTL3-regulated m⁶A targets [MeRIP-Seq] were defined in this study, whereas host-virus physical interactions, effect of genetic perturbation on SARS-CoV-2 replication/infection, and G9a/GLP substrate definitions were curated manually from literature sources.

FIGS. 5A-5B. The enzymatic inhibition of G9a or Ezh2 mitigated overexpression or secretion of COVID-19-characteristic proteins. FIG. 5A. Differentially expressed proteins induced by G9a- or Ezh2-inhibitor at ET condition. T-test p-values were generated from three technical replicates. Proteins with log 2 (fold-change) beyond 0.30 or below −0.30 with p value lower than 0.05 were considered as significantly differential expression. Number of significantly down- and up-regulated proteins were shown on lower panel. Proteins labeled in blue were up-regulated in ET cells compared to naïve cells (N). FIG. 5B. Immunoblot analysis of G9a- or Ezh2-inhibitory changes of SPP1, a COVID-19-characteristic protein.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, the terms “treating,” “treatment”, and “to treat” are used to indicate the production of beneficial or desired results, such as to alleviate symptoms, or eliminate the causation of a disease or disorder either on a temporary or a permanent basis, slow the appearance of symptoms and/or progression of the disorder, or prevent progression of disease. For methods of prevention, a subject to be administered the dietary formulation is generally a subject at risk for an inflammatory condition due to genetic predisposition, diet, exposure to disorder-causing agents, exposure to pathogenic agents, and the like. The term “treat” or “treatment” refer to both therapeutic treatment and prophylactic or preventative measures, wherein the object is to prevent or slow down the development or spread of disease or symptoms. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total). “Treatment” can also refer to prolonging survival as compared to expected survival if not receiving treatment.

The term “subject”, “individual”, and “patient” are used interchangeably herein, and refer to an animal, especially a mammal, for example a human, to whom treatment, with a composition as described herein, is provided. The term “mammal” is intended to encompass a singular “mammal” and plural “mammals,” and includes, but is not limited: to humans, primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears.

In some embodiments, the subject to be used in accordance with the presently disclosed subject matter is a subject in need of treatment and/or diagnosis. In some embodiments, a subject can have or be believed to a chronic inflammation-associated disease, condition or phenotype, including an infection of a COVID virus such as SARS-CoV-2.

As used herein, the terms “inhibit”, “suppress”, “repress”, “downregulate”, “loss of function”, “block of function”, and grammatical variants thereof are used interchangeably and refer to an activity whereby the activity of a biological component, e.g. enzyme, cellular signal, metabolic element, and the like, is greatly or substantially reduced, minimized or blocked as compared to its normal activity (e.g. by 50% or more, or about 50%, 60%, 70%, 80%, 90%, 95%, 99% or more).

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand”, “coding sequence” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Also encompassed are any and all nucleotide sequences that encode the disclosed amino acid sequences, including but not limited to those disclosed in the corresponding GENBANK® entries.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

The terms “modulate” or “alter” are used interchangeably and refer to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the terms “modulate” and/or “alter” can mean “inhibit” or “suppress”, but the use of the words “modulate” and/or “alter” are not limited to this definition.

As used herein, the terms “inhibit”, “suppress”, “repress”, “downregulate”, “loss of function”, “block of function”, and grammatical variants thereof are used interchangeably, and with respect to genes, refer to an activity whereby gene expression (e.g., a level of an RNA encoding one or more gene products) is reduced below that observed in the absence of a composition of the presently disclosed subject matter. In some embodiments, inhibition results in a decrease in the steady state level of a target RNA. By way of example and not limitation, histone methyltransferases, such as G9a, can suppress transcription of a number of genes below that observed in the absence of histone methyltransferases.

The term “RNA” refers to a molecule comprising at least one ribonucleotide residue. By “ribonucleotide” is meant a nucleotide with a hydroxyl group at the 2′ position of a D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

The term “transcription factor” generally refers to a protein that modulates gene expression, such as by interaction with the cis-regulatory element and/or cellular components for transcription, including RNA Polymerase, Transcription Associated Factors (TAFs), chromatin-remodeling proteins, reverse tet-responsive transcriptional activator, and any other relevant protein that impacts gene transcription.

The term “promoter” defines a region within a gene that is positioned 5′ to a coding region of a same gene and functions to direct transcription of the coding region. The promoter region includes a transcriptional start site and at least one cis-regulatory element. The term “promoter” also includes functional portions of a promoter region, wherein the functional portion is sufficient for gene transcription. To determine nucleotide sequences that are functional, the expression of a reporter gene is assayed when variably placed under the direction of a promoter region fragment.

The terms “active”, “functional” and “physiological”, as used for example in “enzymatically active”, “functional chromatin” and “physiologically accurate”, and variations thereof, refer to the states of genes, regulatory components, chromatin, etc. that are reflective of the dynamic states of each as they exists naturally, or in vivo, in contrast to static or non-active states of each. Measurements, detections or screenings based on the active, functional and/or physiologically relevant states of biological indicators can be useful in elucidating a mechanism, or defining a disease state or phenotype, as it occurs naturally. This is in contrast to measurements taken based on static concentrations or quantities of a biological indicator that are not reflective of level of activity or function thereof.

As used herein, the terms “antibody” and “antibodies” refer to proteins comprising one or more polypeptides substantially encoded by immunoglobulin genes or fragments of immunoglobulin genes. The presently disclosed subject matter also includes functional equivalents of the antibodies of the presently disclosed subject matter. As used herein, the phrase “functional equivalent” as it refers to an antibody refers to a molecule that has binding characteristics that are comparable to those of a given antibody. In some embodiments, chimerized, humanized, and single chain antibodies, as well as fragments thereof, are considered functional equivalents of the corresponding antibodies upon which they are based. In some embodiments, the presently disclosed subject matter provides methods for identifying, characterizing and/or developing disease-related components of a gene-specific chromatin regulatory protein complex, wherein one or more antibodies can be used directly, or in assays related thereto, in the identification, characterization and/or isolation of such components.

The term “substantially identical”, as used herein to describe a degree of similarity between nucleotide sequences, peptide sequences and/or amino acid sequences refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide identity, when compared and aligned for maximum correspondence, as measured using a sequence comparison algorithm or by visual inspection. As used herein, the terms “detectable moiety”, “detectable label”, and “detectable agent” refer to any molecule that can be detected by any moiety that can be added to a chemoprobe, antigen, inhibitor, marker, reagent and/or antibody, or a fragment or derivative thereof, that allows for the detection of the chemoprobe, antigen, inhibitor, marker, reagent and/or antibody, fragment, or derivative in vitro and/or in vivo. Representative detectable moieties include, but are not limited to, chromophores, fluorescent moieties, radioacite labels, affinity probes, enzymes, antigens, groups with specific reactivity, chemiluminescent moieties, and electrochemically detectable moieties, etc. In some embodiments, the antibodies are biotinylated.

### II. Pharmaceutical Compositions

The compounds disclosed herein can be formulated in accordance with the routine procedures adapted for a desired administration route. Accordingly, in some embodiments, the presently disclosed subject matter provides a pharmaceutical composition comprising a therapeutically effective amount of a compound as disclosed herein (e.g., G9a inhibitor or EZH2 inhibitor), or a pharmaceutically acceptable salt or solvate thereof, and a pharmaceutically acceptable carrier. The therapeutically effective amount can be determined by testing the compounds in an in vitro or in vivo model and then extrapolating therefrom for dosages in subjects of interest, e.g., humans. The therapeutically effective amount should be enough to exert a therapeutically useful effect in the absence of undesirable side effects in the subject to be treated with the composition.

Pharmaceutically acceptable carriers are well known to those skilled in the art and include, but are not limited to, from about 0.01 to about 0.1M and preferably 0.05M phosphate buffer or 0.8% saline. Such pharmaceutically acceptable carriers can be aqueous or non-aqueous solutions, suspensions and emulsions. Examples of non-aqueous solvents suitable for use in the presently disclosed subject matter include, but are not limited to, propylene glycol, polyethylene glycol, vegetable oils such as olive oil, and injectable organic esters such as ethyl oleate. Aqueous carriers suitable for use in the presently disclosed subject matter include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the presently disclosed subject matter can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Parenteral carriers suitable for use in the presently disclosed subject matter include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the presently disclosed subject matter can be mixed as needed with disintegrants, diluents, granulating agents, lubricants, binders and the like using conventional techniques known in the art. The carriers can also be sterilized using methods that do not deleteriously react with the compounds, as is generally known in the art. The compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Further, formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compounds can further be formulated for topical administration. Suitable topical formulations include one or more compounds in the form of a liquid, lotion, cream or gel. Topical administration can be accomplished by application directly on the treatment area. For example, such application can be accomplished by rubbing the formulation (such as a lotion or gel) onto the skin of the treatment area, or by spray application of a liquid formulation onto the treatment area.

In some formulations, bioimplant materials can be coated with the compounds so as to improve interaction between cells and the implant.

Formulations of the compounds can contain minor amounts of wetting or emulsifying agents, or pH buffering agents. The formulations comprising the compound can be a liquid solution, suspension, emulsion, tablet, pill, capsule, sustained release formulation, or powder.

The compounds can be formulated as a suppository, with traditional binders and carriers such as triglycerides.

Oral formulations can include standard carriers such as pharmaceutical grades of mannitol, lactose, starch, magnesium stearate, polyvinyl pyrollidone, sodium saccharine, cellulose, magnesium carbonate, etc.

In some embodiments, the pharmaceutical composition comprising the compound of the presently disclosed subject matter can include an agent which controls release of the compound, thereby providing a timed or sustained release compound.

An effective amount of the compounds disclosed herein comprise amounts sufficient to produce a noticeable effect, such as, but not limited to, a reduction or cessation of self-administration of alcohol or another substance of abuse, weight loss, lack of weight gain, etc.). Actual dosage levels of active ingredients in a therapeutic compound of the presently disclosed subject matter can be varied so as to administer an amount of the active compound that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

The therapeutically effective amount of a compound can depend on a number of factors. For example, the species, age, and weight of the subject, the precise condition requiring treatment and its severity, the nature of the formulation, and the route of administration are all factors that can be considered. In some embodiments, the therapeutically effective amount is in the range of about 0.1 to about 100 mg/kg body weight of the subject per day. In some embodiments, the therapeutically effective amount is in the range of from about 0.1 to about 20 mg/kg body weight per day. Thus, for a 70 kg adult mammal, one example of an actual amount per day would be between about 10 and about 2000 mg. This amount can be given in a single dose per day or in a number (e.g., 2, 3, 4, or 5) of sub-doses per day such that the total daily dose is the same. The effective amount of a salt or solvate thereof can be determined as a proportion of the effective amount of the compound per se.

A compound of the presently disclosed subject matter can also be useful as adjunctive, add-on or supplementary therapy for the treatment of the above-mentioned diseases/disorders. Said adjunctive, add-on or supplementary therapy means the concomitant or sequential administration of a compound of the presently disclosed subject matter to a subject who has already received administration of, who is receiving administration of, or who will receive administration of one or more additional therapeutic agents for the treatment of the indicated conditions, for example, one or more known anti-depressant, anti-psychotics or anxiolytic agents.

### III. Methods and Compositions for Treating COVID-19 Patients

As the systemic cytokine profiles in severe COVID-19 patients showed similarities to profiles in macrophage activation syndromes, particularly viral sepsis, the mechanistic causes of SARS-CoV-2-induced immunopathology in macrophages was investigated. Macrophage cells that had acquired endotoxin (lipopolysaccharide) tolerance (ET) were used. ET is the common immunopathological background of COVID-19 vulnerable groups that have pre-existing chronic inflammatory diseases. Genome-wide CRISPR screening revealed the crucial role of epigenetic regulation involving chromatin remodeling and histone modification in SARS-CoV-2 infection. Correspondingly, the histone methyltransferases G9a and G9a-like protein (GLP; hereafter G9a will represent both proteins) that are constitutively activated in ET showed upregulated expression in COVID-19 patients with high viral load. Further, this research showed that inhibition of G9a enzyme activity mitigated or reversed ET, which implicated active G9a in ET-related, SARS-CoV-2-induced pathogenesis. Thus, to dissect G9a-associated pathways and mechanisms in ET, chromatin activity-based chemoproteomic (ChaC) approach, which consists of a biotinylated G9a inhibitor UNC0965, was used to capture ET-phenotypic G9a protein complexes and identify their constituents by mass spectrometry (MS). Notably, ChaC-MS is superior to conventional immunoprecipitation-MS that captures protein complexes based only on epitope abundance. ChaC-MS identified protein partners of constitutively active G9a that together represent the ET phenotype. The ET macrophage partners of constitutively active G9a included many proteins associated primarily with translation initiation and elongation, RNA modification and processing, and ribosome biogenesis. Strikingly, these G9a interactors involving translational regulation were also identified in cellular pathways that are upregulated or ‘reshaped’ by SARS-CoV-2. Notably, most cofactors of the (m⁶A) RNA methylase METTL3 were among these ET-phenotypic G9a interactors. METTL3 appears to promote translation of specific oncogenes, and is implicated in inflammation. Thus, without being bound by any particular theory or mechanism of action, the results herein are the first to indicate that G9a exerts a noncanonical (nonepigenetic silencing) function, in conjunction with METTL3 and other translation regulators to promote translation of mRNAs that establish the ET phenotype. As the first step to characterize this novel function of G9a, m⁶A RNA immunoprecipitation and label-free quantitative proteomics was used to identify subsets of m⁶A-tagged mRNAs whose translation showed a dual dependence on G9a and METTL3 in ET macrophages. Among these subsets, the sepsis-characteristic overexpression of an immune checkpoint protein CD274 (programmed cell death-ligand 1 or PD-L1) and multiple chemokine receptors were co-upregulated by G9a and METTL3. Crucially, depletion of G9a and METTL3 restored T cell function and reduced the survival of macrophages. Further, proteome-wide translatome changes following pharmaceutical inhibition or genetic knock-out of G9a was determined. This analysis revealed that, from a broader view extended from the G9a-METTL3 translation regulatory axis, G9a activated the translation of specific proteins that were identified in diverse SARS-CoV-2 upregulated pathways or in the PBMCs or sera of severe COVID patients. Accordingly, treatment of ET macrophages with a G9a inhibitor mitigated the elevated expression of COVID-19-characteristic proteins. Additionally, because the histone methyltransferase Ezh2 was identified as an ET-specific interactor of G9a, ET macrophages were treated with an Ezh2 inhibitor and similarly observed mitigation of elevated COVID-19-characteristic proteins.

From a systems view, it was discovered that the G9a-associated mechanism of SARS-CoV-2 immunopathogenesis in which constitutively active G9a promotes the translation of genes that comprise diverse pathways involved in host-virus interactions or viral replication and the host response to SARS-CoV-2 infection. Thus, G9a-target therapy for COVID-19 has potential multifaceted effects to inhibit host hyperinflammation, restore T cell function, and inhibit virus replication. Thus, proteins showing G9a- or Ezh2-dependent overexpression could serve as biomarkers for stratification of COVID-19 patients responsive to G9a- or Ezh2-target therapy. Our discovery of the mechanism by which the G9a/Ezh2 inhibitors reverse SARS-CoV-2 dysregulated inflammation will facilitate design of effective, precision therapies that may improve or facilitate patient survival or recovery from COVID-19 and other chronic inflammatory diseases.

Thus, provided herein in some embodiments are methods of treating a subject suffering from symptoms related to a coronavirus infection, the method comprising administering to the subject a therapeutically effective amount of an inhibitor of G9a, an inhibitor of Ezh2, and/or combinations thereof. The subject can be suffering from a COVID-19 viral infection, and more particularly SARS-CoV-2 pathologic pathways related to a host response and viral replication from the coronavirus infection or COVID-19 viral infection. The subject can be suffering from a hyperinflammatory response mediated by SARS-CoV-2-dysregulated macrophage activation resulting in a cytokine storm.

As disclosed herein, the G9a inhibitor can comprise any small molecule capable of substantially reducing and/or inhibiting translational regulatory processes associated with G9a. By way of example and not limitation, such reduction or inhibition can in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99%, as compared to G9a activity not reduced/inhibited. The same levels of reduction or inhibition can apply to EZH2 via the disclosed EZH2 inhibitors.

By way of example and not limitation, the G9a inhibitor can comprise UNC0642, and the EZH2 inhibitor can comprise UNC1999.

During treatment a subject in need can be co-administered both the inhibitor of G9a and the inhibitor of Ezh2, by any suitable route of administration as disclosed herein. The administration of the inhibitor of G9a and/or the inhibitor of Ezh2 can reduce and/or inhibit coronavirus replication and/or infection in the subject. The administration of the inhibitor of G9a and/or the inhibitor of Ezh2 restores T cell function to overcome lymphopenia, mitigates hyperinflammation, and/or suppresses of viral replication in the subject. The administration of the inhibitor of G9a and/or the inhibitor of Ezh2 suppresses a systemic hyperinflammatory response in the subject by simultaneously inhibiting multiple components of a COVID-19 cytokine storm, wherein the components of the COVID-19 cytokine storm that are inhibited are ARDS-related proteins and/or sepsis-related proteins, optionally wherein the ARDS-related proteins and/or sepsis-related proteins are selected from the group consisting of SPP1, CCL2, IL1RN, CXCL2, SQSTM1, ANPEP, PLAU, PELI1, PROCR, DST, and FABP4. Notably, sepsis and ARDS are the leading complications associated with mortality and morbidity of COVID-19.

The inhibitor of G9a or Ezh2 can be administered to the subject in a pharmaceutically acceptable formulation or carrier.

For applications other than coronavirus treatments, also provided herein are methods of blocking G9a translational regulation of inflammation in a subject. Such methods can comprise administering to the subject an inhibitor of G9a, wherein G9a translational regulation of inflammation in the subject is blocked or substantially reduced. The subject can be suffering from an infection or other condition causing chronic or acute inflammation, including for example a COVID-19 viral infection. The administration of the inhibitor of G9a can also block METTL3-mediated translational regulation of chronic inflammation in the subject.

Based on the mechanism of action as elucidated herein, also provided are methods of identifying a compound to treat and/or prevent chronic and/or acute inflammation in a subject, the methods comprising identifying a compound that blocks G9a translational regulation of inflammation in a subject. Such identified compounds can comprise an inhibitor of G9a, optionally a small molecule inhibitor.

Provided are also uses of a composition for treating a subject suffering from symptoms related to a coronavirus infection, comprising administering to the subject the composition comprising an inhibitor of G9a, an inhibitor of Ezh2, and/or combinations thereof.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Materials and Methods

Chemicals and Reagents.

Cell culture media, other components, and fetal bovine serum were obtained from Gibco. Trypsin was purchased from Promega. LPS (Escherichia coli 0111:B4, ultrapure) was purchased from InvivoGen (USA, Invivogen, cat #tlrl-pelps). HEK 293 stable TLR4-MD2-CD14 cell line was also purchased from InvivoGen. All chemicals were HPLC-grade unless specifically indicated. Raw264.7 and THP1 cells were purchased from ATCC (Manassas, VA). UNC0642 (G9a inhibitor) and UNC1999 (EZH2 inhibitor)⁶⁰ were synthesized. TMT 11-plex isobaric labeling reagent kit was purchased from Thermo Fisher (Cat. A34808). Antibodies against G9a (07-551) and H3K9me2 (07-441) were from Millipore; Antibodies against EIF4E(11149-1-AP), EIF3B(10319-1-AP), HNRNPA2B1(14813-1-AP), METTL3 (15073-1-AP), YTHDF2(24744-1-AP), PD-L1(66248-1-1g), CDC20(10252-1-AP), CDCA7(15249-1-AP), BIRC5(10508-1-AP), TPX2(11741-1-AP), TOP2A(24641-1-AP), NUSAP1(12024-1-AP), SPP1(22952-1-AP), SQSTM1(18420-1-AP), ACTIN(60008-1-1g) were from Proteintech. Antibody against p-p65 (S536)(3033) was from Cell Signaling. Antibody against Brg1 (H-88) (sc-10768x) was from Santa Cruz. Anti-HA (clone HA-7) and anti-flag M2 (clone M2) antibodies were from Sigma. m6A RNA methylation quantification kit was from Abcam(ab185912).

Cell Lines and Treatment

Raw264.7 cells were cultured in DMEM medium. The human monocytic cell line THP-1 was maintained in RPMI 1640 medium (Gibco). All media were supplemented with 10% fetal bovine serum, 100 U/ml penicillin and streptomycin. Cells were grown at 37° C. in humidified air with 5% carbon dioxide. For ChaC pull-down experiments, Raw264.7 cells were either unstimulated (‘N’) or subjected to a single LPS stimulation with 1 μg/ml (NL′) or first primed with 100 ng/ml LPS to induce endotoxin tolerance for 24 h (‘T’), followed by a second LPS challenge at 1 mg/ml (‘TL’). For the condition requiring G9a inhibitor treatment, 1 μM UNC0642 was added at the time of cell plating.

For global protein profiling, Raw264.7 cells were pre-treated with 0.1 μg/ml LPS for 24 hours, then inhibitor UNC1999 (1 μM) or UNC0642 (1 μM) was added. The cells were collected after inhibitor treatment for 8 h, 24 h and 48 h.

For MeRIP-Seq, THP-1 cells were first incubated in the presence of 60 nM PMA overnight to differentiate into macrophages followed by 48 h resting in PMA-free medium. Cells were either left unstimulated (‘N’) or subjected to a single LPS stimulation at 1 μg/ml (NL′), or first primed with 100 ng/ml LPS to induce endotoxin tolerance for 24 h (‘T’), followed by the second LPS challenge at 1 μg/ml (‘TL’).

UNC0965 Pull-Down and ChaC Sample Processing.

Similar to our recent report,¹⁶ 1 mg nuclear protein extracted from Raw 264.7 macrophage cells was incubated overnight at 4° C. with 2 nmole UNC0965 pre-coupled to 50 μl neutravidin-agarose (Thermo Fisher), and washed three times with 1 ml lysis buffer to remove non-specific bound proteins. For on-beads sampling and processing, five additional washes with 50 mM Tris-HCl pH 8.0, 150 mM NaCl were used to remove residual detergents. On-beads tryptic digestion was performed with 125 μl buffer containing 2 M urea, 50 mM Tris-HCl pH 8.0, 1 mM DTT, 500 ng trypsin (Promega) for 30 min at room temperature on a mixer (Eppendorf). The tryptic digests were eluted twice with a 100 μl elution buffer containing 2 M urea, 50 mM Tris-HCl pH 8.0, 5 mM iodoacetamide. Combined eluates were acidified with trifluoroacetic acid at final concentration of 1% (TFA, mass spec grade, Thermo Fisher) and desalted with a C18 stage tip.

Sample Preparation for TMT Based Global Proteomic Profiling.

Cell pellets were resuspended in 8 M Urea, 50 mM Tris-HCl pH 8.0, reduced with dithiothreitol (5 mM final) for 30 min at room temperature, and alkylated with iodoacetamide (15 mM final) for 45 min in the dark at ambient temperature. Samples were diluted 4-fold with mM Tris-HCl pH 8.0, 1 mM CaCl₂ and digested with trypsin at 1:100 (w/w, trypsin: protein) ratio overnight at room temperature. Peptides were desalted on homemade C18 stage tips. Each peptide sample (100 μg) was labeled with 100 μg of TMT reagent following the optimal protocol⁷⁰. The mixture of labeled peptides was desalted and fractionated into 12 fractions in 10 mM TMAB containing 5-40% acetonitrile.

Polysome Fractionation

METTL3 KO, G9A KO, and Control Raw 264.7 cells were cultured in 10 cm plates until 80% confluent at the time of harvest. Cells were treated with 100 μg/ml cycloheximide (CHX; Sigma) for 10 min at 37° C. Media were removed and the cells were washed twice with 10 ml PBS containing 0.1 mg/ml CHX, scraped, pelleted by spinning for 10 min at 2200 rpm at 4° C. After removing the supernatant, the cells were resuspended with 1 ml lysis buffer (20 mM Tris-HCl, pH 7.4, 140 mM KCl, 5 mM MgCl₂, 1% Triton X-100, 10 mM DTT) containing mg/ml CHX and swelled on ice for 10 min followed by passing through a 27 gauge needle times to break the cell membrane. Spin down the lysate at max speed for 10 min, the supernatant were carefully layered onto 10-50% sucrose gradients and centrifuged at 32,000 rpm (no brake) in a Beckman SW-40 rotor for 2 h at 4° C. Gradients were fractionated and monitored by absorbance 254 nm.

Transfection and CRISPR Knockout

293TLR4-MD2-CD14 cells were seeded in 6-well plates for 24 h before transfection, and the constructs were transfected or co-transfected into MCF7 cells using reagent jetPRIME (Polyplus). After 24 h, the cells were lysed directly in the plates by adding SDS-PAGE sample buffer, heating at 95° C. for 5 min, and sonicating for 5 seconds to clear the lysate for immunoblotting.

For the CRISPR/Cas9 constructs, oligonucleotides for the sgRNA of human and mouse G9a and METTL3 as described below, were annealed and cloned in BsmBI-digested lentiCRISPRv2 (Addgene plasmid #52961). The empty vector was used as a negative control. Viral production was performed with a standard protocol. In brief, a total of 10 μg of plasmid, including target plasmid, pMD2.G (Addgene plasmid #12259) and psPAX2 (Addgene plasmid #12260) with a ratio of 10:5:9, was co-transfected into 293T cells with jetPRIME™. Virus-containing media were collected 48 hours after transfection. MDA-MB-231 cells at 60-80% confluency were incubated with the virus containing media for 24-48 hours, and then subjected to 1.0 μg/mL puromycin selection. After 4-7 days puromycin selection, the stably transfected cells were collected for further analysis.

RNA Isolation and RT-PCR and m6A RNA Methylation Level

Total RNA was isolated using RNeasy kit (Qiagen). First-strand cDNA was synthesized by M-MLV reverse transcriptase (Promega, car #M170B) and diluted by a factor of 10 for quantitative PCR. Real-time PCR was performed using SYBR Green Master Mix (Thermo Fisher, cat #0221). All measurements were normalized to GAPDH and represented as relative ratios. PCR primers for real-time PCR are summarized in Supplementary Table. The m6A level of isolated RNA was measured by using the m6A RNA methylation quantification kit.

Cell Cycle and FACS Analysis

For flow cytometry analyses of cell cycle, cells were incubated with 10 uM EdU (Santa Cruz, sc-284628) for 30 minutes before harvesting by trypsinization. Cells were washed with PBS, and fixed with 4% paraformaldehyde (Electron Microscopy Sciences, 1571s) in PBS for min at room temperature. Then 1% BSA-PBS was added and the cells were centrifuged. Fixed cells were permeabilized with 0.5% Triton X-100 in 1% BSA-PBS at room temperature for 15 min, then centrifuged. Cells were processed for EdU conjugation with 1 μM AF647-azide (Life Technologies, A10277) in 100 mM ascorbic Acid, 1 mM CuSO4, and PBS for 30 minutes at room temperature in the dark. Lastly, cells were washed and incubated in 1 μg/mL DAPI (Life Technologies, D1306) overnight at 4° C. Samples were run on an Attune NxT (Beckman Coulter) and analyzed with FCS Express 7 (De Novo Software).

P14 CD8+ T Cell Proliferation and Activation

P14 CD8+ T cell proliferation and activation assay was performed as described previously⁴⁴. Briefly, CD8+ T cells from a P14 transgenic mouse were first isolated with CD8a microbeads according to manufacturer's instruction. Isolated CD8+ T cells were resuspended in 1 mL 1640 medium and either labeled with 1 mL of the 10 μM Carboxyfluorescein diacetate succinimidyl ester (CFSE) for 8 min at RT for proliferation assay or kept unlabeled for activation assay. METTL3 KO, G9a KO, and control Raw 264.7 cells were seeded in 6-well plates with indicated treatments followed by 50 μg/mL Mitomycin C treatment for 30 min at 37° C. Cells were washed with 1 mL PBS twice and then labeled with 0.4 mL of the 30 μg/mL GP33-41 peptide in PBS for 30 min at 37° C. One hundred thousand P14 T cells were co-cultured with 2×10⁵ control, METTL3 KO, and G9a KO cells in 96-well plates in RPMI medium containing 50 U/mL mIL-2. The proliferation and activation of CD8+ T cells were assessed by flow-cytometry at day six.

LC-MS/MS Analysis

For ChaC pull-down samples, desalted peptide mixtures were dissolved in 30 μl 0.1% formic acid (Thermo Fisher). Peptide concentration was measured with Pierce™ Quantitative Colorimetric Peptide Assay (Thermo Fisher). In the Easy nanoLC-Q Exactive HFX setup, peptides were loaded on to a 15 cm C18 RP column (15 cm×75 μm ID, C18, 2 μm, Acclaim Pepmap RSLC, Thermo Fisher) and eluted with a gradient of 2-30% buffer B at a constant flow rate of 300 nl/min for 30 min followed by 30% to 45% B in 5 min and 100% B for 10 min. The Q-Exactive HFX was also operated in the positive-ion mode but with a data-dependent top 20 method. Survey scans were acquired at a resolution of 60,000 at m/z 200. Up to the top 20 most abundant isotope patterns with charge≥2 from the survey scan were selected with an isolation window of 1.5 m/z and fragmented by HCD with normalized collision energies of 27. The maximum ion injection time for the survey scan and the MS/MS scans was 100 ms, and the ion target values were set to 3e6 and 1e5, respectively. Selected sequenced ions were dynamically excluded for 30 seconds.

For global protein profiling, 0.5 μg of each fraction was analyzed on a Q-Exactive HF-X coupled with an Easy nanoLC 1200 (Thermo Fisher Scientific, San Jose, CA). Peptides were loaded on to a nanoEase MZ HSS T3 Column (100 Å, 1.8 μm, 75 μm×150 mm, Waters). Analytical separation of all peptides was achieved with 100-min gradient. A linear gradient of to 10% buffer B over 5 min, 10% to 31% buffer B over 70 min and 31% to 75% buffer B over 15 minutes was executed at a 300 nl/min flow rate followed a ramp to 100% B in 1 min and 9-min wash with 100% B, where buffer A was aqueous 0.1% formic acid, and buffer B was 80% acetonitrile and 0.1% formic acid.

Peptides were separated with 45-min gradient, a linear gradient of 5 to 30% B over 29 min, 30 to 45% B over 6 min followed a ramp to 100% B in 1 min and 9-min wash with 100% B. LC-MS experiments were also performed in a data-dependent mode with full MS (externally calibrated to a mass accuracy of <5 ppm and a resolution of 120,000 for TMT-labeled samples or 60,000 for secretome samples at m/z 200) followed by high energy collision-activated dissociation-MS/MS with a resolution of 45,000 for TMT-labeled global samples and 15,000 for secretome samples at m/z 200. High energy collision-activated dissociation-MS/MS was used to dissociate peptides at a normalized collision energy of 32 eV (for TMT-labeled sample) or 27 eV in the presence of nitrogen bath gas atoms. Dynamic exclusion was 45 or 20 seconds. Each fraction was subjected to three technical replicate LC-MS analyses. There were two biological replicates of samples and two technical replicates were executed for each sample.

Proteomics Data Processing and Analysis

Mass spectra were processed, and peptide identification was performed using the MaxQuant software version 1.6.10.43 (Max Planck Institute, Germany). All protein database searches were performed against the UniProt human protein sequence database (UP000005640). A false discovery rate (FDR) for both peptide-spectrum match (PSM) and protein assignment was set at 1%. Search parameters included up to two missed cleavages at Lys/Arg on the sequence, oxidation of methionine, and protein N-terminal acetylation as a dynamic modification. Carbamidomethylation of cysteine residues was considered as a static modification. Peptide identifications are reported by filtering of reverse and contaminant entries and assigning to their leading razor protein. The TMT reporter intensity found in MaxQuant was for quantitation. Data processing and statistical analysis were performed on Perseus (Version 1.6.0.7). Protein quantitation was performed using TMT reporter intensity found in MaxQuant and a one-sample t-test statistics on three technical replicates was used with a p-value of 5% to report statistically significant protein abundance fold-changes. Label-free quantification (LFQ) was for ChaC interactome and secretome data analysis.

Analysis of Functional Category and Networks

The biological processes and molecular functions of the G9a-interacting proteins were categorized by IPA and STRING.

m⁶a RNA Immunoprecipitation Sequencing (MeRIP-Seq) and Data Analysis

m⁶A-RIP-Seq was performed as described previously with slight modifications 71. Messenger RNA from 10 ug total RNA extracted from Ctrl, METTL3 KO and G9a KO cell samples was purified with Dynabeads Oligo (dT)₂₅ (Thermo Fisher; 61006). Ten percent of 150 ng mRNA was used as Input mRNA, and the remainder was incubated with 3 ug anti-m⁶A polyclonal antibody (Synaptic Systems; 202003) which was preconjugated to Dynabeads Protein A (Thermo Fisher; 10001D) in 500 uL IP buffer (50 mM Tris, pH 7.4, 150 mM NaCl, Igepal CA-630) for 2 hours at 4° C. After washing twice with IP-buffer and twice with High-Saltwash buffer (50 mM Tris pH 7.4, 500 mM NaCl, 0.1% Igepal CA-630) for 5 minutes each, the m6A mRNA was eluted with 100 uL IP-buffer containing 6.7 mM N⁶-Methyladenosine (Sigma-Aldrich; M2780) and 40 U RNase Inhibitor (NEB, M0314S) and then recovered with RNA Clean and Concentrator-5 spin columns (Zymo; R1015).

### Artificially Synthesized Primer Nucleotide

The Input mRNA and m⁶A-IPed mRNA were subjected to library generation using the SMART-seq protocol as described (Full-length RNA-seq from single cells using Smart-seq2. Picelli et al., 2014). For the synthesis of the first strand cDNA, the mRNA was mixed with 0.25 μL RNase inhibitor and 1 μL CDS primer (SEQ ID NO. 1: 5′-AAGCAGTGGTATCAACGCAGAGTACT30VN-3′) and heated to 70° C. for 2 min. Then the mixture containing 0.5 μL of 100 mM DTT, 0.3 μL of 200 mM MgCl₂, 1 μL of 10 mM dNTPs, μL RNase inhibitor, 1 μL of 10 μM TSO primer (SEQ ID NO. 2: 5′-AAGCAGTGGTATCAACGCAGAGTACATrGrGrG-3′), 2 μL of 5× SMARTScribe RT buffer and 0.5 μL SMARTScribe reverse transcriptase (Takara, 639536) was added for performing reverse transcription. The cDNA was then amplified by Advantage Polymerase Mix (TAKARA, 639201) with IS primer (SEQ ID NO. 3: 5′-AAGCAGTGGTATCAACGCAGAGT-3′). After purification with 0.8× AMPure XP beads (Fisher Scientific, A63880), the fragmentation of 100 pg cDNA was performed with EZ Tn5 Transposase (Lucigen, TNP92110). Fragments of cDNA were amplified by KAPA HiFi hotstart readymix (EMSCO/FISHER, KK2601) with the Nextera i7 primer and Nextera i5 primer. The DNA was purified with 0.8× AMPure XP beads and quantified by qPCR with KAPA Library Quantification Kit (Fisher, NC0078468). The DNA from different samples was pooled at equal molar amounts, and the final sequencing library was loaded at concentrations of 2.7 pM, and sequenced on a NextSeq 550 (Illumina) for single-read sequencing.

The raw sequencing data were demultiplexed with bc12fastq2 v2.17.1.14 (Illumina) and the adapter was trimmed by Trimmomatic-0.32 software (Trimmomatic: a flexible trimmer for Illumina sequence data. Bolger et al., 2014). Then the Input and m6A-IP reads were mapped to human genome version hg38 by STAR v.2.5.2a (STAR: ultrafast universal RNA-seq aligner; Dobin et al., 2012), and only uniquely mapping reads at the exon level for each gene were quantified and summarized to gene counts, which were further analyzed in R v.3.6.2. After normalization, sorting, and indexing with Samtools-1.1 software, the corresponding BAM files for each sample were loaded to IGV software to generate the peaks plots.

AACT Pulse-Labeling and Measurements of Protein Turnover Rates

G9a KO and control Raw 264.7 cells were cultured in AACT/SILAC DMEM medium supplemented with regular lysine and arginine (KORO) and 10% dialyzed fetal bovine serum (Thermo Fisher), 1% penicillin, and streptomycin for five passages. G9a KO, control and (1 μM) UNC0642 treated control cells were either untreated (N) or treated with low dose (100 ng/ml) LPS to induce endotoxin tolerance. The cells were washed with PBS twice to remove light medium (KORO), and then switched to heavy AACT DMEM medium containing stable isotope-enriched D4-lysine and ¹³C₆-arginine to label newly synthesized proteins. The cells were harvested at 2 h, 4 h, 8 h, 24 h, 48 h, and 72 h, and lysed in 8 M urea containing 50 mM Tris-HCl pH 8.0. One hundred micrograms protein from each condition was digested with trypsin, desalted, and fractionated with C18 material (High pH) into eight fractions followed by LC-MS/MS analysis.

Underlying Mathematical Model for Protein Kinetics and Interpretation of Offset (γ)

Mathematical equations used to model synthesis and degradation of proteins in cell-lines/conditions with different doubling times were inspired from mathematical models described previously⁷³⁻⁷⁶ and adapted to yield interpretable analytical solutions for our system. Briefly, it was assumed that there is a source of amino acids and a pool of degraded waste products, both of which contain medium and heavy amino acids. Assuming a switch from medium to heavy label (i.e., M→H medium switch at t=0), the heavy source pool was assumed to be the inexhaustible medium in which the cells were grown (t>0). Additionally, there is a contamination of the heavy amino acid source pool via recycling so that fraction γ(t) of the amino acids in source pool is M-labelled. The following items are other assumptions of the model include:
a) Proteins are synthesized at a constant rate (S) using medium and heavy amino acids from the source pool. Therefore, synthesis is a zero-order process with respect to protein concentration
b) Proteins are degraded at constant rate (k) into the degradation pool. Probability of a protein being degraded is the same for old and newly synthesized protein molecules and remains constant during the lifetime of these proteins
c) Rate of cell division (k_{div}) is constant (for each cell-line/condition) and contributes to overall degradation and synthesis rates (i.e., a protein with degradation rate of 0 will still show a loss of pre-existing M label because of its distribution equally to daughter cells. Similar thing occurs for newly synthesized proteins as intensity of H-label will increase owing to cell doubling even if protein's per cell concentration is constant)
d) Cells are at steady state. The concentration per cell of any given protein remains constant during the experiment. Therefore, synthesis and degradation rates of a protein are equal (i.e., S=k+k_{div}) and first order labelling kinetics were adopted for curve fitting and intensities were fitted to exponential equations.

All model equations/diagrams are for a protein i with the index dropped for clarity. Master equations describing the system are as follows:

$$\frac{d M}{d t} = S γ ( t ) - ( k + {k}_{div} ) M \qquad ( 1 )$$

$$\frac{d H}{d t} = S [ 1 - γ ( t ) ] - ( k + {k}_{div} ) H$$

where M=M (t) and H=H (t) are dimensionless abundances of medium and heavy proteins, respectively.

Constant Offset (γ):

In steady state expect the contamination fraction to stabilize at a certain level, γ(t)=γ=const. In such case the equations (1) have a trivial solution:

$$M ( t ) = {C}_{1} {e}^{- ( k + {k}_{div} ) t} + \frac{S γ}{( k + {k}_{div} )} \qquad ( 2 )$$

$$H ( t ) = {C}_{2} {e}^{- ( k + {k}_{div} ) t} + \frac{S ( 1 - γ )}{( k + {k}_{div} )}$$

A stationary solution is required where the total amount of protein is constant, H (t)+M (t)=const. Hence,

$$\frac{d}{dt} ( H + M ) = S γ - ( k + {k}_{div} ) M + S ( 1 - γ ) - ( k + {k}_{div} ) H = 0$$

and we find S=(k+k_{div})(H+M). If the data us normalized, such that H (t)+M (t)=1 it is found that:

S=(k+k_{div}) (3)

At time t=0 we have H(t)=Hₒ and M(t)=Mₒ. Therefore, using equations (2) and (3), it is found C₁ and C₂ and plugging their value back into equations in (2), we get:

M(t)=(Mₒ−γ)e^{-(k+k}^{_{div}}^{)t}+γ

H(t)=(1−γ)(1−e^{-(k+k}^{_{div}}^{)t})+Hₒe^{-(k+k}^{_{div}}^{)t} (4)

Where Mₒ and Hₒ are intensities of medium and heavy labeled proteins at t=0 and should ideally be 1 and 0 respectively (assuming M→H label switch at t=0). However, they have been left as free variables in the equations above to account for offset (γ) caused by amino acid recycling, experimental variation, and errors in data fitting. The degradation curve M(t) follows a simple exponential decay with an offset (γ) which corresponds to the (constant) fraction of contamination of the amino acid source pool.

Analytical solution for determining protein half-life from equations in (4) is as follows:

$${t}_{1 / 2} ( Deg ) = - \frac{ln ( \frac{{M}_{0} - 3 γ}{2 ( {M}_{0} - γ )} )}{k - {k}_{div}} ; {t}_{1 / 2} ( Syn ) = - \frac{ln ( \frac{1 - γ}{2 ( 1 - γ - {H}_{0} )} )}{k - {k}_{div}} \qquad ( 5 )$$

However, because of errors while fitting free variables (γ, k and H₀ or Mₒ), the solutions are not stable. Therefore, a numerical solution is required to determine protein half-life instead. Numerical solutions show that in the initial part of the decay, γ(t) is small and S≈k+k_{div}. Hence, equations can be rewritten in (4):

$$\frac{d M}{d t} = S γ ( t ) - ( k + {k}_{div} ) M \approx ( k + {k}_{div} ) ( 0 ) - ( k + {k}_{div} ) M \approx - ( k + {k}_{div} ) M \qquad ( 6 )$$

$$\frac{d H}{d t} = S [ 1 - γ ( t ) ] - ( k + {k}_{div} ) H \approx ( k + {k}_{div} ) - ( k + {k}_{div} ) H \approx ( k + {k}_{div} ) ( 1 - H )$$

These are trivial equations of pure degradation and synthesis with exponential solutions of the form (at t=0) as shown:

M(t)=M₀e^{-(k+k}^{_{div}}^{)t}

H(t)=1−e^{-(k+k}^{_{div}}^{)t} (7)

From this an approximate relation between half-life and the degradation/synthesis coefficient is found, given by:

$${t}_{1 / 2} = - \frac{ln ( \frac{1}{2} )}{( k + {k}_{div} )} = \frac{ln ( 2 )}{( k + {k}_{div} )} \qquad ( 8 )$$

Asymptotic Offset (γ):

The constant γ case can be generalized to a scenario in which γ(t) increases from the initial γ(0)=0 until the entire system reaches an asymptotic equilibrium. This was consistent with our data where observed M(t) and H(t) reach an equilibrium for most of the proteins. In this asymptotic offset (γ) state, there is

$$\frac{d M}{d t} = 0 , \frac{d H}{d t} = 0 \qquad ( 9 )$$

$$γ ( t ) = {γ}_{∞} , M ( t ) = {M}_{∞} , H ( t ) = {H}_{∞}$$

Substituting these into the original equations (1) yields

0=Sγ_{∞}−(k+k_{div})M_{∞}

0=S[1−γ_{∞}]−(k+k_{div})H_{∞} (10)

Which gives M _{∞}+H_{∞}=S/(k+k_{div}). Again, a stationary solution with normalization M(t)+=1 is required, hence S=(k+k_{div}). A similar interpretation of the offset is reached: it is explained by the asymptotic contamination fraction, M_{∞}=γ_{∞}.

Exponential Offset (γ):

One possible functional form for γ(t) is an exponential growth, γ(t)=γ_{∞}(1−e^{-kt}). In such case the first equation of (1) assumes the form

$$\frac{d M}{d t} = S {γ}_{∞} ( 1 - {e}^{- ( k + {k}_{div} ) t} ) - ( k + {k}_{div} ) M \qquad ( 11 )$$

which can be solved analytically

$$M ( t ) = {M}_{0} {e}^{- ( k + {k}_{div} ) t} - S {γ}_{∞} t {e}^{- ( k + {k}_{div} ) t} + \frac{S {γ}_{∞}}{( k + {k}_{div} )} \qquad ( 12 )$$

and in a case of balanced synthesis and degradation

(S=(k+k_{div})),M(t)=(M₀−(k+k_{div})γ_{∞}t)e^{-(k+k}^{_{div)t}}+γ_{∞} is obtained. (13)

This is a modified exponential decay with a constant asymptotic offset, γ_{∞}. The shape of this curve resembles a simple exponential decay with an offset. The offset, γ_{∞}, is interpreted again as the asymptotic contamination fraction of the source pool.

AACT/SILAC Pulse-Labeling Data Extraction and Normalization:

Cell lysates were collected at 2 h, 4 h, 8 h, 24 h, 48 h and 72 h from wildtype (Ctrl), G9a knockout (G9a-KO) and 1 μM UNC0642-treated Raw 264.7 cells under N and T conditions, digested with trypsin, fractionated by HPLC and finally subjected to LC-MS/MS analysis. To correct for errors introduced during sample-preparation/data-acquisition, protein intensities were normalized based on the premise that the sum of KO/RO (M) and the K4/R6 (H) intensities should be constant across different time-points. After normalization, curves for estimation of turnover rates, determined from the kinetics of AACT label incorporation or loss, were fitted based on the assumption of exponential protein degradation (or synthesis) and constant offset (γ).

Proteins identified in at-least 4 out of 6 time points were selected for curve fitting using the model described above. The turnover rates (k _{deg}/k _{syn}), curve maxima (M ₒ/H ₒ), offsets (γ) and goodness-of-fit statistics (SSE, RMSE, rsquared, adjrsquared) were obtained for each protein using a nonlinear least square (NLS) method in MATLAB (vR2017b). To remove poor quality quantitative data, different filter criteria for k, R², M ₒ/Hₒ and γ were applied, and curves that were at the border of passing these filter criteria were manually inspected. The final filtering criteria were based on the goal to remove spectra that showed a high variation of data points along the fitted curve (M ₒ/H ₒ and R ²), a high offset (γ), or resulted in turnover rates (K) that simply could not be determined accurately considering the pulse time-points chosen in the experimental design. Eventually, only entries were kept that met following filter criteria: k _{deg}/k_{syn}: [0, 5]; M ₒ: [0.65, 1.5]; H ₒ: [−0.5, 0.50]; γ: [−0.2, 0.5] and coefficient of determination R ²≥0.8. Subsequently, protein synthesis (t_{1/2syn}) and degradation (t_{1/2deg}) half-lives were estimated using equation (8). Finally, synthesis and degradation half-lives were combined to obtain median protein turnover time/half-life (t_{1/2}).

### Example 1

### Constitutively Active G9a is Implicated in SARS-CoV-2 Upregulated Translation Pathways

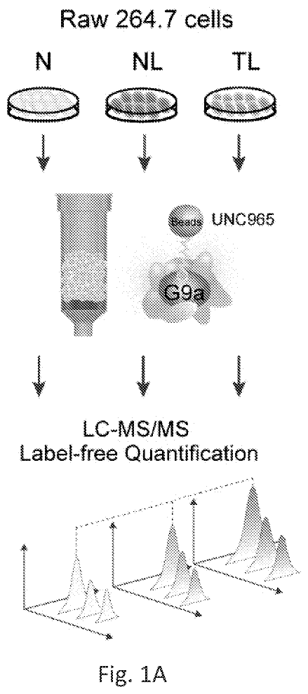

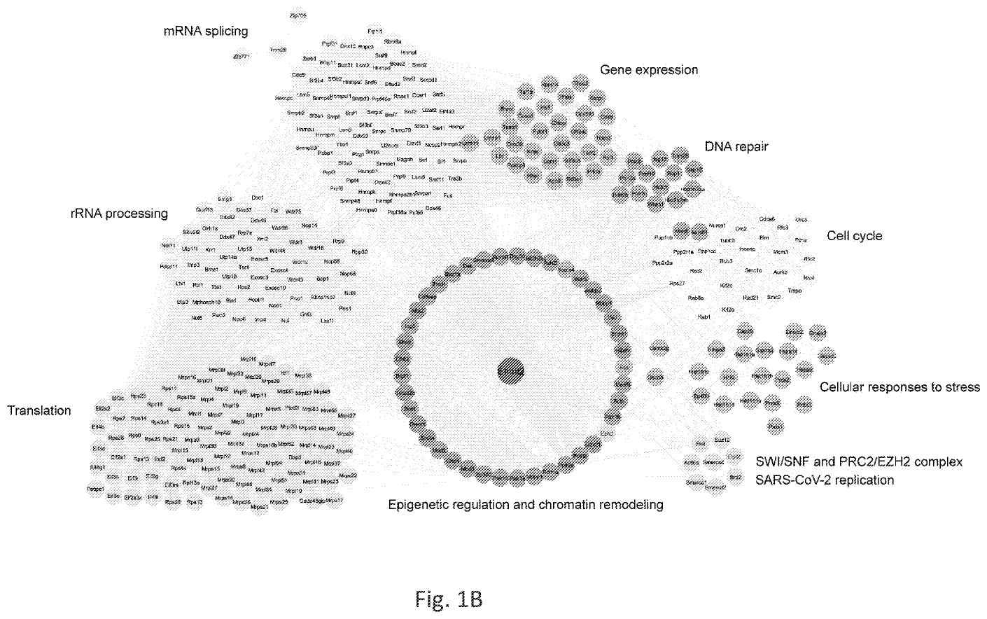

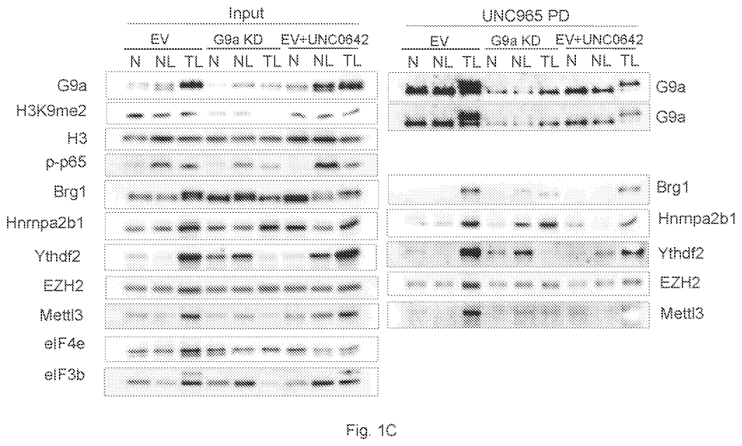

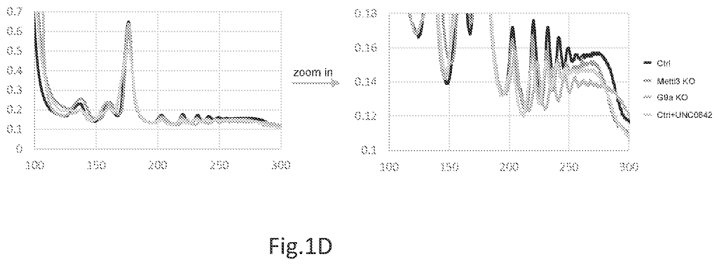

mRNAs which encode major components of the G9a/GLP (EHMT2/EHMT1)-associating complex were found to be overexpressed in COVID-19 patients with increasing SARS-CoV-2 load. Accordingly, by top-down mass spectrometry (MS), ChIP-PCR, and ChaC chemoprobe pull-down, it was found that the methylation activity of G9a was constitutively higher in chronically inflamed or endotoxin-tolerant (TL or ET) macrophages compared with acutely inflamed (NL) cells. Thus, label-free quantitation (LFQ), UNC0965 ChaC experiments were performed on mouse Raw 264.7 macrophages under nonstimulated (N) and different inflammatory conditions (NL, TL) (FIG. 1A). On the basis of LFQ ratios that are proportional to the relative binding of individual proteins to G9a in TL versus NL/N, greater than 1,000 proteins that showed consistently enhanced interaction with G9a in TL/ET macrophages were identified.

STRING (Search Tool for the Retrieval of Interacting Genes) was used to identify enriched functional pathways/processes that were represented by these ET-specific G9a interactors (FIG. 1B). First, in agreement with G9a's canonical epigenetic regulatory function, proteins associated with chromatin remodeling and histone modification were identified. These chromatin proteins included the SWI/SNF remodeling complex and BRD4, which were characterized by siRNA and genome-wide CRISPR screens as essential host factors for SARS-specific and pan-coronavirus infection, respectively (FIG. 1B). These results implicated the ET-phenotypic G9a interactome in SARS-CoV-2 immunopathogenesis. Surprisingly, ChaC data from ET macrophages predicted that, via ET-specific interactions with some representative proteins, G9a may have a noncanonical (nonepigenetic) function in major translation regulatory processes. Strikingly, fifty-three G9a interactors that were identified were found by Bojkova et al. in SARS-CoV-2 upregulated pathways associated with translation initiation/elongation, alterative splicing, RNA processing, nucleic acid metabolism, and ribosome biogenesis. This coincident identification validated the similarity between ET-immunophenotypic G9a pathways and pathways activated by SARS-CoV-2. For example, the splicing factor SF3B1 and the 40S ribosomal protein Rps14 as ET-specific G9a interactors were identified, and Bojkova et al. found that emetine inhibition of Rps14 or pladienolide inhibition of SF3B1 significantly reduced SARS-CoV-2 replication. Like our finding for G9a interactors in cancer patients with poor prognosis, the ChaC-MS results for ET macrophages showed that certain genes upregulated by SARS-CoV-2 infection are fully translated to their encoded proteins as ET-specific G9a interactors. Thus, constitutively active G9a may coordinate these SARS-CoV-2 activated translation pathways.

### Example 2

### Interaction with METTL3 and Other Translation Proteins Implicates G9a in Translational Regulation of Chronic Inflammation

It was noticed that UNC0965 captured most known cofactors of METTL3. METTL3 enhances mRNA translation by interaction with the translation initiation machinery such as NCBP1/2 (CBP80) and RBM15, HNRNPA2B1, eIF3, and eIF4E. Although METTL3 itself was not identified by MS, our identification of METTL3 cofactors as TL-specific G9a interactors indicated that G9a may regulate translation via interaction with METTL3. Most of the other translation regulatory proteins in Flag-tagged METTL3 pull-down from TL macrophages were also identified. For example, ribosomal proteins that bind to actively translated mRNA were identified, including forty-six 39S ribosomal proteins (Mrp11-57), 28S (Mrps), 40S ribosomal proteins (Rps), 60S ribosomal proteins (RP1), 60S ribosome subunit biogenesis protein NIP7 homolog, ribosome-binding protein 1 (Rrbp 1), ribosomal RNA processing proteins (Rrp1, Rrp36, Rrp7a, Rrp8) as well as multiple RNA-binding proteins (FIG. 1B). These results from unbiased interactome screening indicated that G9a and METTL3 function in the same translation regulatory pathways. Immunoblotting showed TL-specific association with endogenous G9a with METTL3 and major translation regulators (FIG. 1C), in agreement with the ChaC result. In macrophages with G9a knock-down, UNC0965 pulled down only proteins that interacted with the residual G9a. These results confirmed the new function of G9a in METTL3-mediated translation in ET macrophages.

Then, by polysome analysis, the effect of G9a and METTL3 on protein synthesis was determined. First, CRISPR/Cas9 was used to knock out (ko) G9a (EHMT2) or METTL3 in macrophages subjected to LPS stimulation to generate cells with different inflammatory conditions (NL, T, or TL). G9a depletion caused reduced protein expression of METTL3 in TL/ET. Also, as indicated by reduced levels of the proinflammatory markers such as phosphor-p65 and phospho-IκB in the macrophages under prolonged LPS stimulation, depletion of either METTL3 or G9a similarly caused reduced ET and resensitized the ET macrophages to LPS stimulation. These results suggested that ET is coregulated by G9a and METTL3. Next, global protein synthesis was measured from polysome abundance in wild type versus G9a ko or METTL3 ko macrophages. FIG. 1D shows that the polysome abundance decreased in G9a ko or G9a inhibitor(UNC0642) treated or METTL3 ko cells compared with abundance in wild type. Thus, depletion of either G9a or METTL3 suppressed protein synthesis that was dependent on G9a and METTL3 in the endotoxin-tolerant macrophages.

### Example 3

### G9a and METTL3 Co-Upregulate Translation of m⁶A-Marked mRNA Subsets

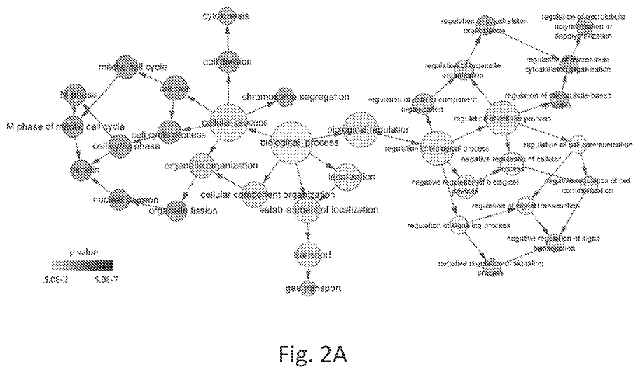

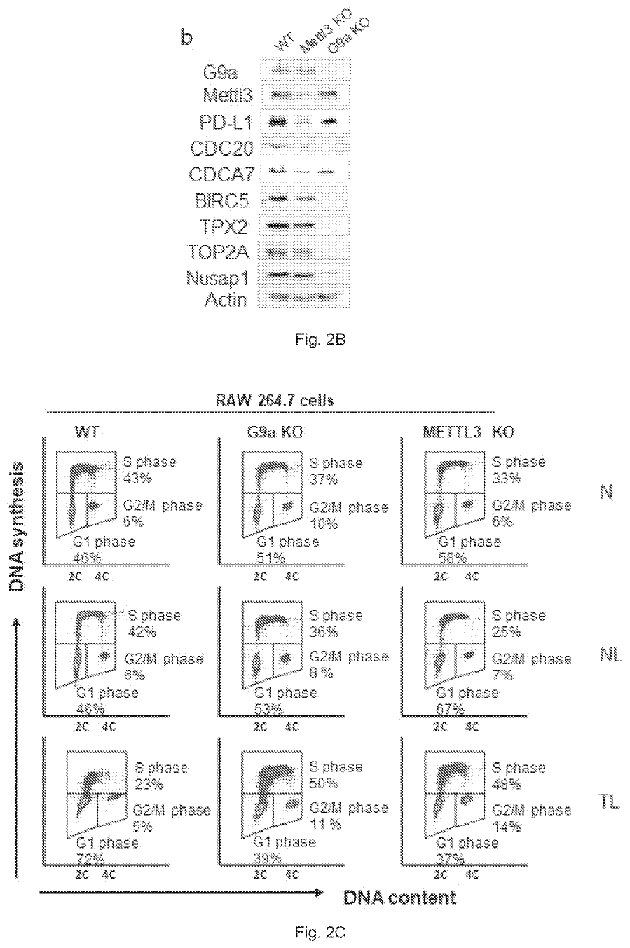

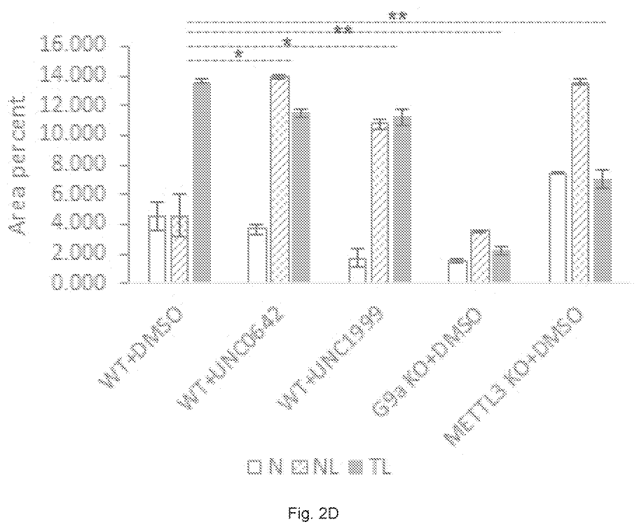

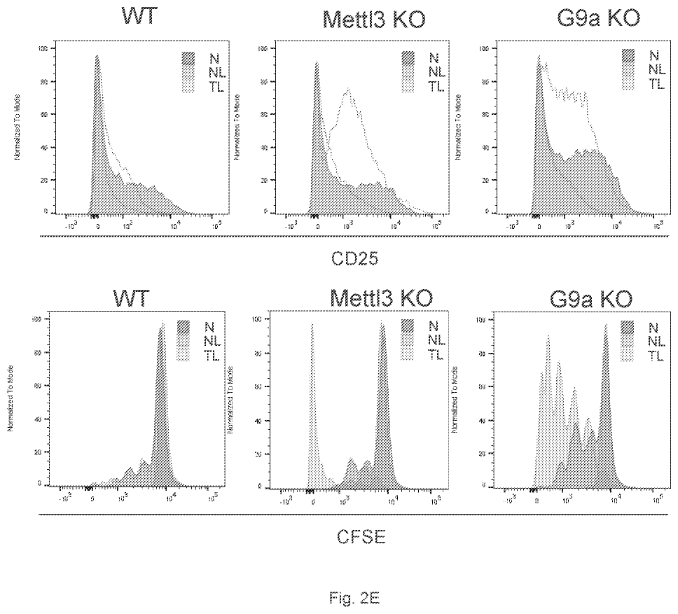

Because global protein synthesis in ET macrophages required G9a and METTL3, experiments were designed to identify specific mRNAs whose translation was dependent on G9a and METTL3. Thus, RNA-seq and m⁶A RNA immunoprecipitation-sequencing (MeRIP-Seq) was performed on wild type, G9a ko, and METTL3 ko THP1 macrophages in N, NL, TL. First, in the G9a-depleted macrophages under TL, 440 immune or inflammatory response genes exhibited increased mRNA abundance. This finding of the G9a-suppressed genes aligned with our previous report that, via interactions with transcriptional repressors such as cMyc, constitutively active G9a suppresses the transcription of proinflammatory genes. Conversely, in both G9a ko and METTL3 ko cells under TL, 136 genes with decreased mRNA expression were identified compared to wild type cells; these genes are associated mostly with regulation of cell cycle progression, cell proliferation, and antiviral or anti-inflammatory responses. Like the co-existence of G9a and METTL3 in TL-specific translation regulatory complexes, this new finding of G9a/METTL3-co-upregulated genes indicated that G9a and METTL3 cooperate to posttranscriptionally activate expression of pro-survival and anti-inflammatory genes in TL. Further, 62 of 136 G9a/METTL3-co-upregulated genes were tagged with m⁶A (FIG. 2A). The Integrative Genomics Viewer plots showed that the m⁶A level of most mRNAs that encode these G9a/METTL3-co-upregulated genes decreased in either G9a ko or METTL3 ko macrophages with TL. Notably, the ‘stabilized’ or ‘actively translated’ mRNA (total input) that was proportional to the amount of m⁶A-tagged transcripts also showed a dependence on G9a and METTL3. The G9a- and METTL3-dependence of the abundance of m⁶A mRNA coding these genes was validated by quantitative PCR.

To determine the predominant mechanism responsible for the G9a-mediated translation of specific genes, it was examined whether G9a ko affected the m⁶A level of total RNA under N, NL, TL. A relatively reduced m⁶A level was observed in G9a ko and METTL3 ko cells. In addition, LFQ proteomics was performed to identify proteins that exhibited G9a- or METTL3-dependent expression changes in the same THP macrophage set (e.g., wild type versus G9a ko versus METTL3). Principle component analysis showed that, in TL, G9a ko or METTL3 ko produced clusters of protein expression profiles that were well separated from the clusters of wild type ET macrophages. This result indicated that depletion of G9a and METTL3 led to characteristic protein expression patterns that represented similar inflammatory phenotypes. Importantly, the G9a/lVIETTL3-co-upregulated m⁶A mRNA or proteins that were identified by nonbiased MeRIP-Seq or LFQ proteomics functionally overlapped with pathways related to antiviral immune response and cell fate determination. Immunoblotting confirmed that the protein expression of select genes in these functional clusters had a dual dependence on G9a and METTL3 (FIG. 2B), which indicated that G9a and METTL3 coactivate translation of the same sets of m⁶A-tagged mRNA. In agreement with our ChaC-MS finding of enhanced interactions of G9a with METTL3 and associated translational regulatory proteins (FIG. 1B), this epitranscriptomic-to-proteomic correlation for specific genes indicated that G9a is directly involved in gene-specific translation regulatory processes in ET macrophages.

### Example 4

### G9a and METTL3 Promote Proliferation of ET Macrophages that Produce Organ-Damaging Inflammatory Factors

One cluster of the proteins co-upregulated by G9a and METTL3, BIRC5, CDC20, NUSAP1, CDCA7, TOP2A, and ALCAM, is functionally associated with cell division, mitotic cell cycle, and cell proliferation (FIG. 2A). This observation not only aligned with the function of METTL3 in promoting cell cycle progression and survival, but, more importantly, the observation implicated constitutively active G9a in promoting METTL3-mediated translation of prosurvival proteins, e.g., METTL3-mediated m⁶A promotes translation of cMyc mRNA and is responsible for cMyc stability in AML cells.

Therefore, flow cytometry was used to determine the effect of G9a and METTL3 on the cell cycle in different inflammatory conditions. As shown in FIG. 2C, conditions that mimicked chronic inflammation (TL/ET) caused a dramatic accumulation of macrophages in G1 compared with nonstimulated (N) or acutely inflamed (NL) conditions (72% vs 46%). This G1 accumulation was accompanied by fewer S phase cells (23% vs 43%). Neither G9a nor METTL3 knockout delayed the G1 phase under the TL condition. Prolonged LPS stimulation (TL) caused these G9a- or METTL3-knockout lines to spend more time in S phase relative to wild type cells and relative to the same cells under acute inflammation conditions. The clonogenic assay (FIG. 2D) showed both knockout lines survived less well than controls during TL treatment, and the G1 delay could serve a protective function. Akin to our proteomic finding that G9a/METTL3 coactivate expression of multiple prosurvival proteins, these cell cycle analyses indicated that constitutively active G9a and METTL3 cooperate to restrain cell cycle progression and promote increased survival of the slow-processing macrophages during chronic inflammation, possibly by targeting prosurvival mRNAs for translation specifically in the growth phase (G1).

### Example 5

### G9a and METTL3 Co-Upregulated ET Overexpression of PD-L1, and Depletion of G9a or METTL3 Restored T Cell Function

Another cluster of G9a/METTL3-co-upregulated proteins, PD-L1, CX3CR1, and IRF8, are functionally associated with immune checkpoint regulation and antimicrobial response. PD-L1 is overexpressed in sepsis patients with impaired T cell function, and, likewise, our MeRIP-Seq data showed an increased level of PD-L1 (CD274) m⁶A mRNA under ET. Although PD-L1 m⁶A mRNA exhibited little dependence on G9a or METTL3 similar to certain METTL3-regulated genes, results from LFQ proteomics and immunoblotting consistently showed that the ET overexpression of PD-L1 was dependent on G9a and METTL3 (FIG. 2B). Thus, this data suggests for the first time that G9a and METTL3 impair T cell function via promoting translation or overexpression of PD-L1 in ET macrophages.

To confirm this, a similar T cell proliferation assay was performed to determine the effect of either G9a ko or METTL3 ko on T cell function under the TL/ET condition. T-cell activation and proliferation were compared by incubation of wild type, G9a ko, or METTL3 ko Raw cells collected in N, NL, and TL with T cells from a P14 transgenic mouse. By monitoring multiple markers of T-cell activation, including CD25, CD44, and CD69, it was observed that the co-existing TL/ET wild type cells suppressed activation of CD8⁺ T cells, whereas incubation with G9a ko or METTL3 ko cells produced efficiently activated T cells (FIG. 2E, upper panel). Similarly, it was found that a greater number of proliferated P14 CD8⁺ T cells after six days of co-incubation of T cells with either G9a ko or METTL3 ko, whereas wild type cells lost the ability to promote proliferation of T cells in ET (FIG. 2E, lower panel). In line with downregulation of proteins that were highly enriched in T cell activation in severe COVID-19 patients, these results showed that G9a and METTL3 impair T cell function by promoting overexpression of immune checkpoint proteins such as PD-L1.

### Example 6

### Lysine Methylation by G9a is Critical for the Stability of METTL3 Complexes in ET Macrophage Gene-Specific Translation

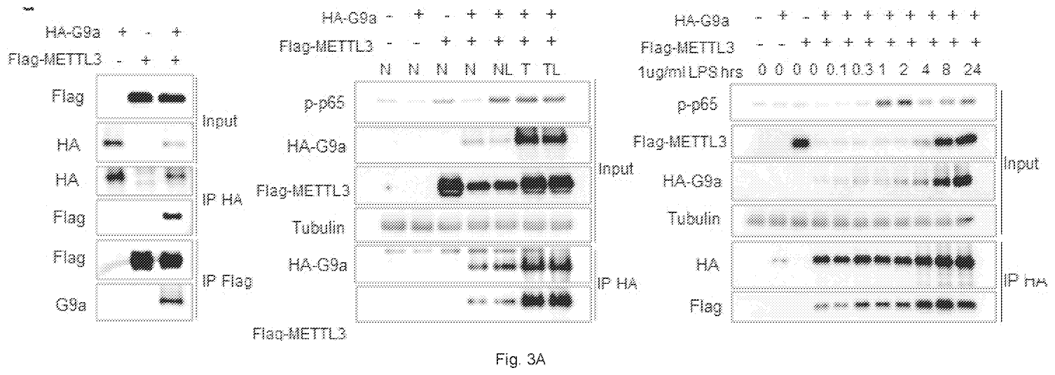

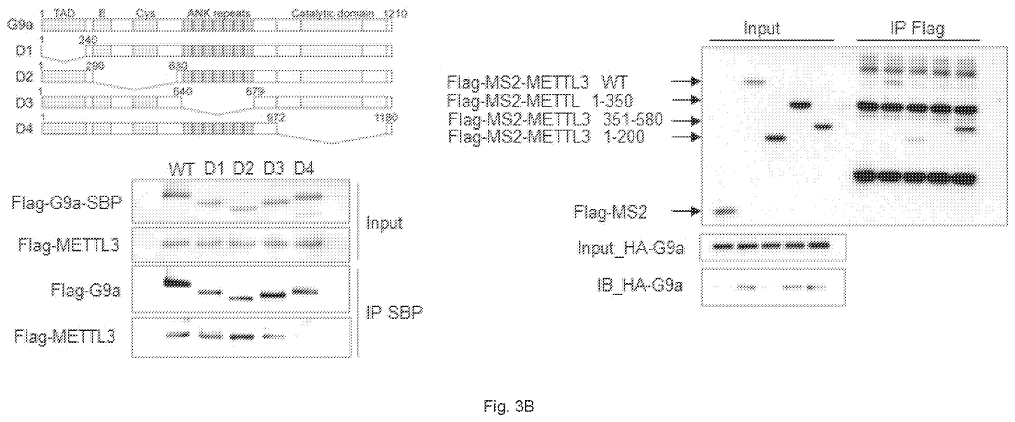

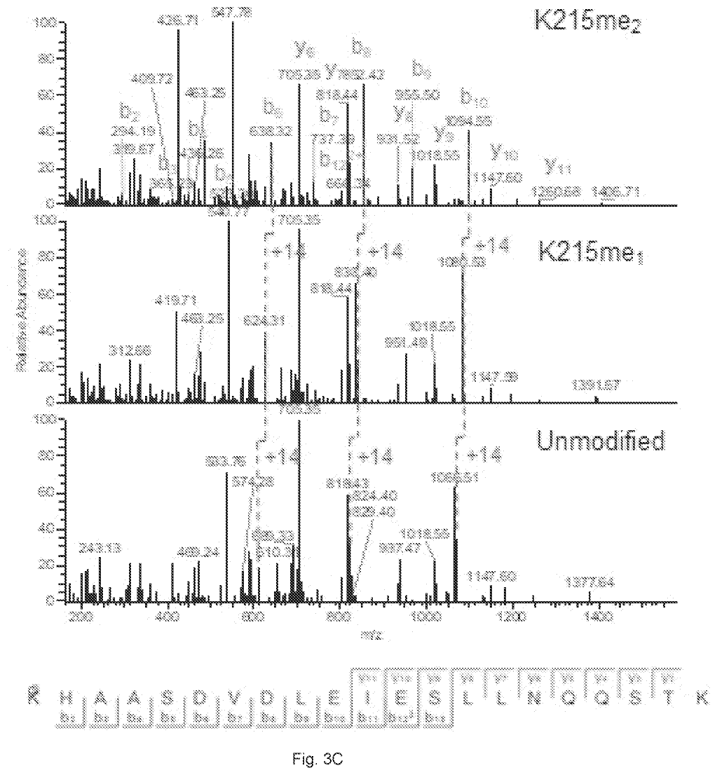

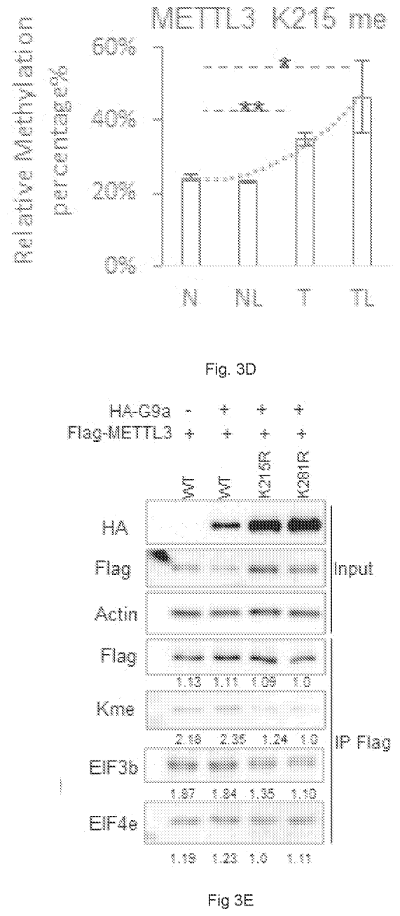

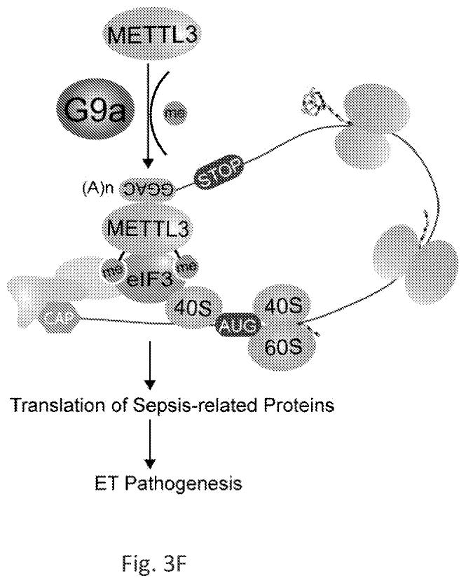

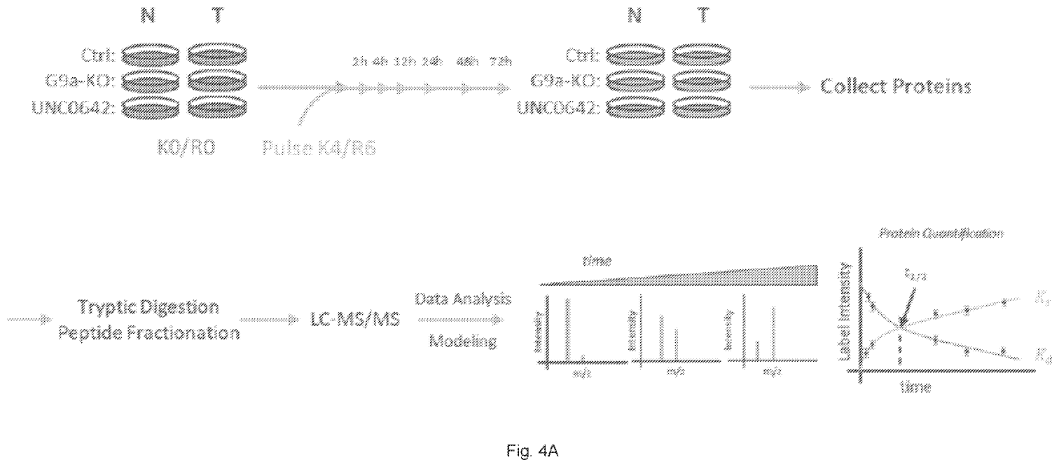

Next, the function of the G9a-METTL3 interaction in ET macrophages was investigated. Briefly, equal amounts of HA-tagged G9a and Flag-tagged METTL3 were co-transfected into the LPS-responsive, TLR4/CD14/1VD2 293 cells under N, NL, or TL. Notably, both METTL3 and G9a (input) showed significantly increased co-expression specifically in T or TL (ET). Immunoprecipitation with anti-HA antibody confirmed TL-specific interaction of the two proteins. Importantly, the METTL3-G9a interactions also increased with prolonged LPS stimulation that mimicked ET (FIG. 3A). Notably, the composition of the UNC0965-captured G9a interactome from ET/TL macrophages showed significant overlap with the composition of the G9a lysine methylation (Kme) proteome; eight nonhistone G9a substrates were identified as ET-specific G9a interactors. These results indicated that certain G9a interactors are ET-specific G9a substrates.

Considering that some G9a interactors are ET-specific G9a substrates, it was examined whether the ET-specific interaction between G9a and METTL3 implied that G9a methylates METTL3 in ET. With domain-truncated versions, it was found that the catalytic domain of G9a interacts with the C-terminus (aa 201-580) of METTL3 (FIG. 3B). Flag-METTL3 was then immunoprecipitated from the cotransfected TLR4/CD14/MD2 293 cells in TL; immunoblotting with an anti-mono- or di-methylysine antibody showed that METTL3 was methylated. LC-MS/MS was then used to sequence the tryptic digests of Flag-METTL3 and unambiguously identified four mono- or di-methyl-lysine (Kme) sites in the C-terminus of METTL3 that directly interact with the G9a catalytic domain (FIG. 3C). In addition, using LFQ MS to measure the differences in these Kme sites in different inflammatory conditions, one Kme site (K215) was identified that showed increased abundance in ET (FIG. 3D), thereby confirming that these METTL3 lysines are targets of constitutively active G9a. Du et al. reported that SUMOylation of METTL3 at lysine 215 modulates the RNA N⁶-adenosine-methyltransferase activity of METTL3. Also, E3 SUMO-protein ligases PIAS1, RanBP2, and CBX4, and a SUMO1-specific protease SENP1, were identified as ET-specific interactors of METTL3. SENP1 reduced METTL3 SUMOylation at multiple lysine sites including K215. These results confirmed the propensity of METTL3 to be post-translationally modified and the functional relevance of distinct modification (e.g., lysine methylation) types in ET macrophages. Notably, the transient nature of enzyme-substrate interaction probably explained why ChaC-MS did not unambiguously identify the endogenous G9a-METTL3 interaction.

Initiation is usually the rate-limiting step in translation, and METTL3 enhances translation of target mRNAs by recruiting eIF3 to the initiation complex. Single and double, nonmethylatable mutants of METTL3, i.e., K215, K281, and K327-to-R (KXR), were made and used immunoblotting to compare the strength of binding of indicated proteins to the Flag-METTL3 versus Flag-K215R or K281R METTL3 mutants in the ET TLR4/CD14/MD2 293 cells. It was observed that Kme absence (confirmed with Kme antibodies) weakened METTL3 interaction with eIF3 by at least 30% (FIG. 3E), a deficiency that was expected to impair METTL3-mediated translation. Notably, the METTL3 interaction with the 5′ cap-bound eIF4e was unaffected by the K215R or K281R mutation, a finding that agreed with the fact that METTL3 is critical for 5′UTR m⁶A mRNA to promote cap-independent translation. A mechanism underlying G9a methylation-activated, METTL3-mediated translation (FIG. 3F) is postulated based on results from the abovementioned combined approaches.

### Example 7

### G9a Coordinates a Widespread Acceleration of Gene-Specific Translation in ET

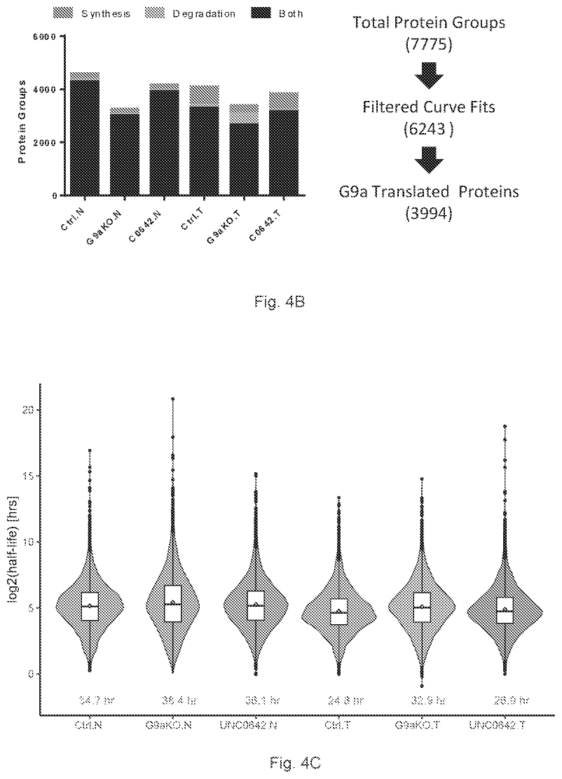

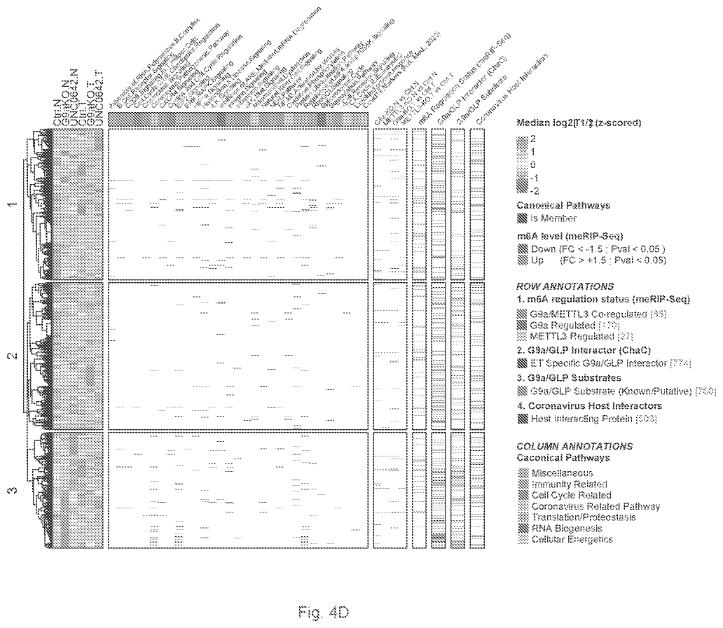

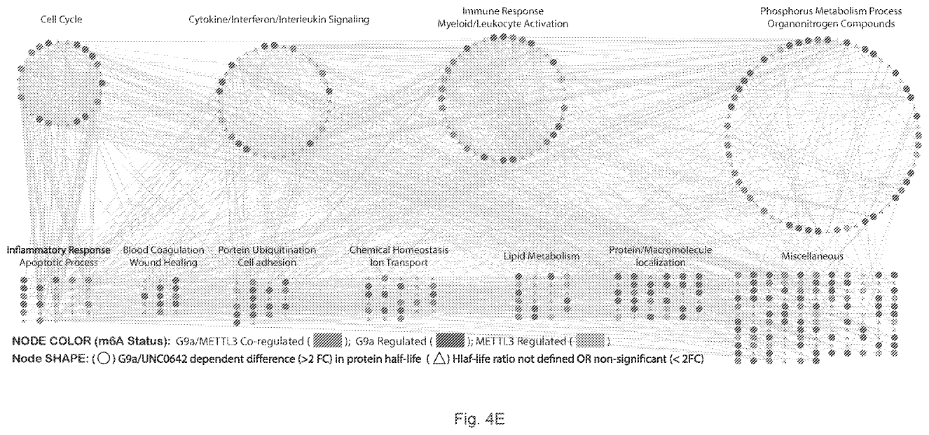

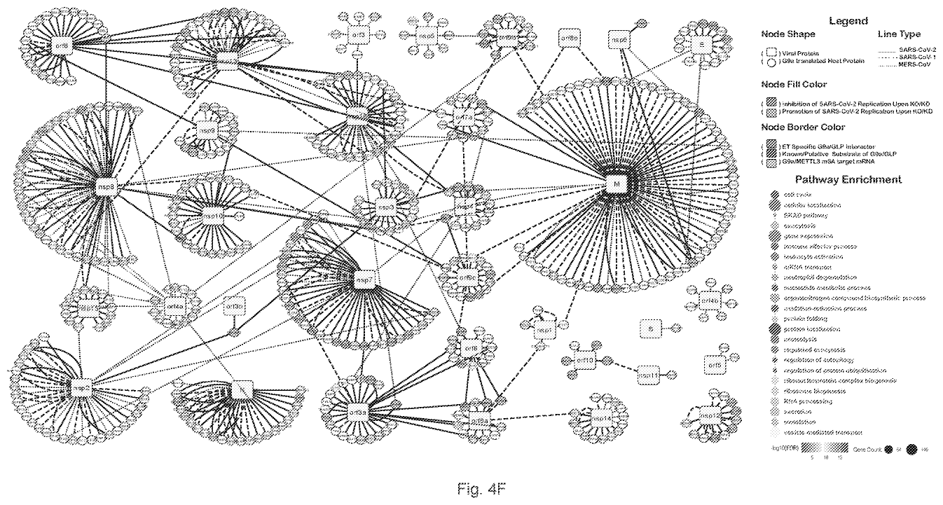

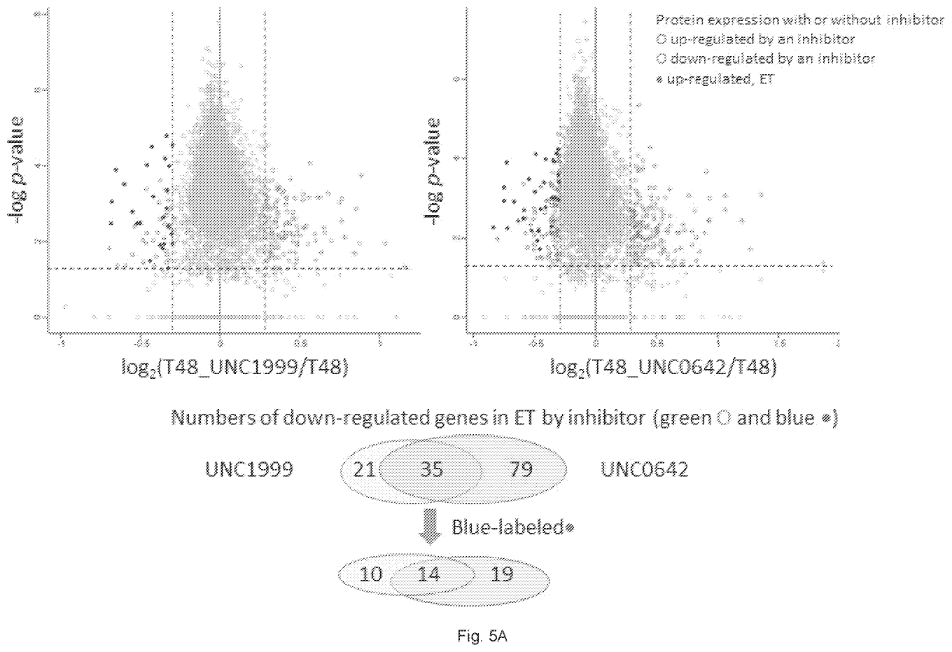

ChaC-MS identifications of additional translation regulators as G9a interactors indicated that the G9a-METTL3 axis is only one part of a translation regulatory network involving G9a (FIG. 1A). Thus, the improved of AACT or SILAC pulse-labeling translatome strategy (FIG. 4A) was employed to profile, in a nonbiased proteome-wide manner, all genes that showed G9a-dependent translation, i.e., the ‘G9a-translated proteins’. Accordingly, individual protein rates of synthesis, degradation, and overall turnover were determined in snapshot samples collected at different times from cultures of wild type, G9a ko, and UNC0642-treated macrophages. Here, genetic (G9a ko) and pharmacologic inhibition (UNC0642 treatment) of G9a decreased synthesis or turnover of G9a-translated proteins.

Briefly, cells grown with Lys0-Arg0 (K0/R0, ‘light’, L) were pulse-labeled with Lys4-Arg6 (K4/R6, ‘medium’, M) supplemented media. At 2 h, 4 h, 8 h, 24 h, 48 h, and 72 h after the media switch, proteins extracted from harvested cells were subjected to tryptic digestion, fractionation, and LC-MS/MS. This experimental design yields (i) increasing signals from the K4R6-labeled protein molecules due to nascent protein synthesis, and (ii) decreasing signals from KORO-labeled proteins due to degradation or secretion of pre-existing protein molecules. Accordingly, the inhibitor-induced rate changes in nascent protein synthesis or protein degradation were quantified by the intensities of L or M labels at different time points in wild type, G9a ko, and UNC0642-treated macrophages, respectively, under non-stimulated (N) or prolonged endotoxin stimulation (T) conditions (FIG. 4A). The model assumed steady-state equilibrium conditions, in which the rate of increase was counterbalanced by the rate of decrease, leading to stable intracellular protein levels. Effects of amino acid recycling and differences in cell division rate between different conditions were also considered. Fit qualities were estimated using least-squared regression (R²), root-mean-squared error (RMSE), with additional thresholds on fitted parameters to ensure good/meaningful estimates of protein turnover.

Synthesis and/or degradation half-lives for 6,243 protein groups in the combined dataset (i.e., wild-type/Ctrl, G9a-KO, UNC0642-treated cells under N and T conditions) were obtained. For 78-94% of fitted proteins, information was available for both AACT-label increase and decrease, which provided an internal duplicate measurement of protein turnover time for each sample in a steady-state system (FIG. 4B). Half-life determination was reliable across labeling pairs (R=0.99) and cell culture replicates (R=0.29-0.99) as evidenced by high Pearson's correlation coefficients and covariance. Under N and T conditions, whereas the principle component analysis hinted towards distinct proteostasis landscapes in macrophages, G9a knock-out or inhibition produced large effects on protein turnover compared with wild type macrophages. Consequently, significant pairwise differences in global protein turnover time upon G9a inhibition as well as ET were observed. Estimated protein turnover times spanned four orders of magnitude (with some outliers), from minutes to thousands of hours. Similar to SARS-CoV-2-upregulated global translation in multiple organs of severe patients, ET also increased the rates of global translation or protein turnover as evidenced by shorter median half-lives in T (24.8-32.9 h) compared with N (34.7-38.4 h). The ET-accelerated translation rates were reduced in G9a ko or UNC0642-treated macrophages specifically under T (32.9, 26.9 h) (FIG. 4C). In agreement with the polysome results that showed G9a/METTL3-dependent protein synthesis (FIG. 1C), this nonbiased translatome profiling indicated that constitutively active G9a accelerated global protein synthesis and degradation (i.e., global proteostasis) in ET.

### Example 8

### Constitutively Active G9a Promotes Translation of Specific Protein Components in SARS-CoV-2 Pathologic Pathways Related to the Host Response and Viral Replication

Among 6,243 proteins with AACT-pulse labeling-measured turnover rates, 3,994 were identified as G9a-translated proteins based on pairwise comparisons of protein half-lives. Pathway enrichment analysis indicated that all G9a-translated proteins are primarily involved in immune responses involving B-cell, T-cell, NK-cell, chemokine, interferon, interleukin signaling, G1/S checkpoint and cyclin signaling, RNA biogenesis such as splicing and mRNA degradation, RNA Pol II assembly, translation/proteostasis including EIF2/4, ubiquitination, SUMOylation, unfolded protein response signaling, cellular energetics including oxidative phosphorylation and TCA cycle signaling, and coronavirus-related pathways. These results showed that, coincident with ET-phenotypic increases in global rates of protein turnover (see FIG. 4C), constitutively active G9a upregulates diverse pathways by activating the translation of particular pathway genes. A closer look at net protein turnover in individual pathways indicated that the global protein turnover trend did not completely capture the underlying complexity of G9a/ET regulated proteostasis because the effects of G9a inhibition on individual pathways varied greatly. FIG. 4D shows a summary heatmap depicting median protein half-lives of G9a-translated proteins and their associated pathways in ET macrophages. Interestingly, of these 3,994 G9a-translated proteins, 774 proteins were identified by UNC0965 ChaC-MS as ET-phenotypic G9a interactors, and 760 proteins are known or putative substrates of G9a/GLP. G9a mediated methylation of a nonhistone substrate FOXO1 has already been shown to induce proteasomal degradation 57. Therefore, our results not only support our identification of METTL3 as both G9a interactor and a nonhistone substrate of G9a (FIG. 3C) but also indicate that G9a may upregulate gene-specific turnover by interacting with or methylating select translation regulators. Notably, 472 G9a-translated proteins were encoded by G9a and/or METTL3-regulated m⁶A mRNAs (FIG. 4D), of which 282 proteins (˜59.7%) showed more than two-fold difference in turnover time in a G9a-dependent manner (FIG. 4E). These m⁶A mRNA-encoded, G9a-translated proteins are associated with cell cycle, cytokine/chemokine/interleukin signaling, myeloid/leukocyte activation, blood coagulation and wound healing, proteostasis including ubiquitination, localization (i.e. endocytosis, vesicle transport) signaling, organo-nitrogen metabolism, i.e., lipid, nucleotide, amino-acid synthesis, and ion transport (FIG. 4E). Thus, combined results from ChaC-MS, m⁶ARIP-Seq, and translatome analysis validated the regulatory function of the G9a-METTL3-m⁶A axis in ET-phenotypic translation activation, which accounted for a subset of G9a-translated genes (472 out of 3994). Clearly, via interactions with distinct translation regulators other than METTL3 (FIG. 1B), G9a coordinates additional, as yet unknown, mechanisms to facilitate gene-specific translation (FIG. 4D).

Strikingly, almost all of the G9a-translated pathways that were identified by translatome profiling (FIGS. 4D and 4E) have been implicated in SARS-CoV-2 life cycle and COVID-19 pathogenesis. Indeed, G9a-dependent turnover for 11 COVID-19 markers, 503 SARS-CoV-1/2 & MERS-CoV host interactors (FIG. 4F) and 66 other coronavirus pathogenesis pathway-related proteins were observed. Several of these G9a-translated proteins were identified as ET-specific G9a interactors, nonhistone G9a substrates or G9a/METTL3-dependent m⁶A targets, which supported the translation regulatory function of G9a in COVID-19 pathogenesis. More importantly, genetic perturbation of several of these G9a translated host interactors adversely affected SARS-CoV-2 replication and infection (FIG. 4F). Similarly, several host factors critical for SARS-CoV-2 infection identified in siRNA/CRISPR based screens are closely related to G9a complex. In line with COVID-19 hallmarks of a systemic cytokine storm, excessive infiltration of monocytes, dysregulated macrophages, and impaired T cells, faster turnover of proteins that belong to immune response pathways involving B-cell receptor, T-cell receptor, NK-cell, chemokine, interferon, interleukin, Jak/Stat, NF-kB and CXCR4 signaling were observed. Further, pharmacologic inhibition of G9a downregulated these pathways by reducing the translation/turnover rates of major pathway components. Similarly, proteins involved in splicing, unfolded protein response, and translation initiation/elongation were upregulated following SARS-CoV-2 infection. Consistent with these findings, increased turnover was observed for greater than 150 proteins related to translation/proteostasis including EIF2/4, unfolded protein response, SUMOylation and ubiquitination signaling and RNA biogenesis such as spliceosomal cycle and RNA degradation pathways in ET macrophages, whereas G9a inhibition reversed these effects by reducing their turnover times. In short, constitutively active G9a regulates specific genes at the translational or posttranslational level to drive ET-related, SARS-Cov-2-induced pathogenesis, and inhibition of G9a and its associated proteins hinders coronavirus replication and infection. Thus, G9a and its associated proteins are potential drug targets to treat COVID-19 and other coronavirus-related ailments, and these targets merit further molecular and clinical study.

### Example 9

### Enzymatic Inhibition of G9a or Ezh2 Similarly Mitigated Overexpression of COVID-19-Characteristic Proteins

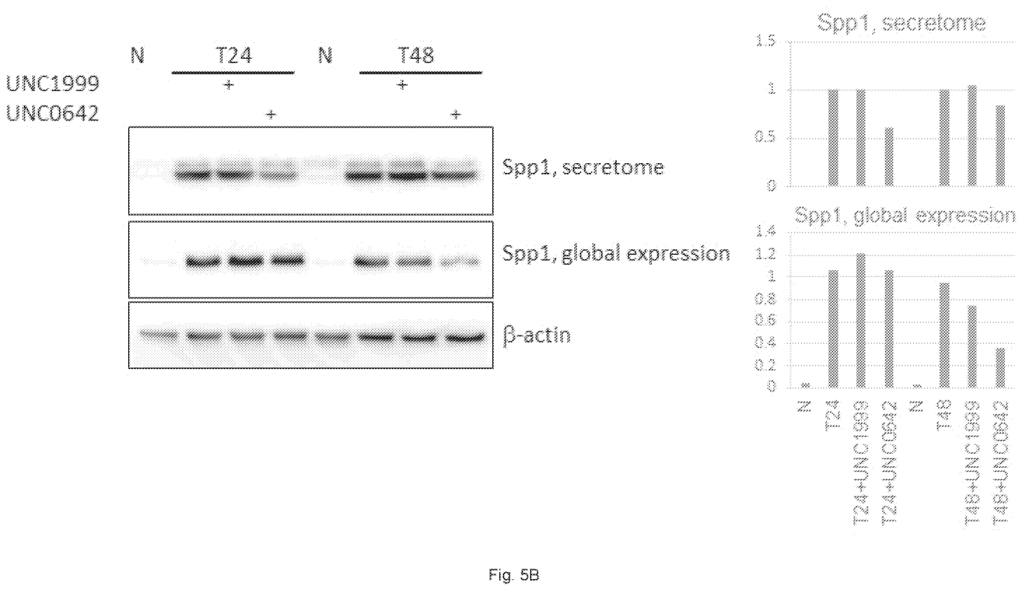

Interestingly, the histone methyltransferase Ezh2 (Enhancer of zeste homolog 2) and two Polycomb Repressive Complex 2 (PRC2) components, SUZ12 and EED were also identified as ET-specific interactors of G9a (FIG. 1B). Further, the inventors were intrigued by the G9a-dependent translation of the PRC2 complex components, which includes Ezh2, in ET. Like the elevated level of G9a mRNA, most PRC2 complex components were overexpressed in COVID-19 patients with high viral load. Because there are clinically validated inhibitors of Ezh2 in antitumor clinical trials, Ezh2 inhibitors were considered for severe COVID-19 therapy and compared the proteomic effects of an Ezh2 inhibitor (UNC1999) with a G9a inhibitor. Various quantitative proteomic approaches, i.e., a multiplex TMT quantitative proteomic method to analyze the intracellular proteins collected from the pellets of N, NL, and T Raw macrophages, were used with or without inhibitor treatment. In parallel, LFQ proteomic approach was used to comparatively identify the proteins whose secretion showed dependence on the treatment by either G9a or Ezh2 inhibitor or both, respectively. Based on their overexpression in ET macrophages, 43 proteins (FIG. 5A) whose abundances were suppressed by either or both inhibitors were identified. Strikingly, >80% of these proteins that showed G9a- or Ezh2-dependent overexpression in ET macrophages were associated with ARDS-related cytokine storm or clinical ARDS, with some proteins found in the sera of severe COVID patients (FIG. 5B).

### Example 10

### Discovery of Nonepigenetic Function of G9a in Driving SARS-CoV-2 Immunopathogenesis

In 10-20% of patients, SARS-CoV-2 infection progresses to ARDS or/and severe pneumonia or multi-organ damage/failure; most of these patients are vulnerable because of pre-existing chronic conditions. The hyperinflammatory response mediated by SARS-CoV-2-dysregulated macrophage activation results in a cytokine storm, which is the major cause of disease severity and death. Also, the immunologic characteristics of COVID-19, especially in severe cases that require ICU care, include reduced counts of CD3+, CD4+ and CD8+T lymphocytes or lymphopenia, and significantly increased levels of certain serum cytokines or hyperinflammation. In addition, as indicated by significantly higher levels of a T cell exhaustion marker PD-1, the surviving T cells in severe patients appeared to be functionally exhausted. A new gene-specific translation mechanism of hyperinflammation, lymphopenia and viral replication was discovered in which the constitutively active G9a/GLP interactome coordinates the networked, SARS-CoV-2-dysregulated pathways that determine COVID-19 severity.

Endotoxin-tolerant macrophages have molecular characteristics similar to chronic inflammation-associated complications (e.g., ARDS and sepsis), including downregulation of inflammatory mediators and upregulation of other antimicrobial factors. These complications systemically contribute to impaired adaptive immunity (e.g., T-cell function impairment or a poor switch to the adaptive response) and susceptibility to secondary infection with an organ-damaging cytokine storm. The current model for inflammation control in ET macrophages was derived from mRNA expression studies. Foster et al. reported that the chromatin modification landscape was differentially programmed in a gene-specific (pro-inflammatory versus anti-microbial genes) manner. That is, with prolonged LPS stimulation, the promoter chromatin of pro-inflammatory genes transitioned from transcriptionally activate to transcriptionally silent and LPS-induced transcripts regulated increased expression of other antimicrobial genes in ET. In parallel with G9a's canonical epigenetic function for transcriptional silencing of pro-inflammatory genes, particular G9a interactors associated with chromatin regulation such as BRD2/4 were found to affect SARS-CoV-2 replication. Additional G9a interactors including the components of chromatin remodeling complexes such as SMARCA2/4, SMARCC2, CREBBP, were characterized as proviral host factors essential for SARS-CoV-2 infection (FIG. 1C). Conversely, upon restimulation, antimicrobial proteins are unlikely to be derived from time-consuming transcription processes as the turnover of mRNAs is an energy-cost-effective process to respond to an infection, which suggests that overexpression of antimicrobial genes in ET is regulated by under-characterized translation mechanisms. Coincident with its constitutive activity in ET and overexpression in COVID-19 patients with high viral load, here G9a is characterized as a noncanonical (nonepigenetic) regulator of gene-specific translation to drive SARS-CoV-2 immunopathogenesis. Mechanistically, via ET-phenotypic interactions with METTL3, a known gene-specific translation activator, and other translation regulators, G9a activates diverse translation regulatory pathways associated with major clinical characteristics of COVID-19. Specifically, on the basis of results from MS/MS-sequencing and biochemical assays, the promoting activity of the G9a-METTL3 axis in gene-specific translation, in which G9a-mediated methylation enhances the stability of the METTL3/m⁶A-mediated translation regulatory complex was clarified (FIG. 3F).

From a broad view, SARS-CoV-2 infection upregulates G9a/GLP which ‘reshapes’ the translation regulatory pathways and, in turn, activates the translation of a range of genes for SARS-CoV-2 immunopathogenesis. Briefly, Bojkova et al. reported that SARS-CoV-2 infection led to increased expression of proteins associated with translation initiation and elongation, alternative splicing, mRNA processing, and nucleic acid metabolism. Likewise, in the ET macrophages that showed similar immunologic features to severe COVID-19, it was found that most of these SARS-CoV-2-upregulated translation components in infected cells were ET-phenotypic interactors and nonhistone substrates of constitutively active G9a (FIG. 4). Further, by nonbiased screening of the ET macrophage translatome with time-dependent inhibition of G9a, genes that are translated at higher rates in a G9a-dependent manner as ‘G9a-translated proteins’ were identified. Clustered by functional pathway enrichment, −4,000 G9a-translated proteins were merged into two major networks associated, respectively, with the host response to SARS-CoV-2 infection (FIG. 4E) or host-virus interactions (FIG. 4F). For example, COVID-19 severity was linked to multiple SARS-CoV-2-induced, G9a-translated host response pathways, primarily associated with immune complement and coagulation dysregulation, IFNs- and IL-6-dependent inflammatory responses, and ER-associated degradation (ERAD) (FIG. 4E). The G9a-translated components of complement and coagulation pathways including C5aR1, SERPINE1, CR1L were upregulated in severe patients. Overexpression of the C5a-05aR1 axis was associated with ARDS in COVID-19 patients. Specifically, increased PD-L1 levels in monocytes and dendritic cells and elevated levels of C5aR1 in blood and pulmonary myeloid cells contribute to COVID-19-characteristic hyperinflammation and ARDS. G9a inhibition reduced the turnover rates of both proteins in ET macrophages. These COVID-19-associated networks composed of G9a-translated proteins provide a compelling rationale to suspect that G9a inhibition will adversely affect SARS-CoV-2-upregulated pathways associated with not only the impaired host response but also with viral infection and replication.

### Example 11

### Inhibitor Mechanism of Action for Severe COVID-19 Therapeutics

Once immunopathologic complications such as ARDS or pneumonia occur, particularly in patients with pre-existing chronic inflammatory diseases, antiviral treatment alone is less effective and should be combined with appropriate anti-inflammatory treatment. However, current immunomodulatory measures failed to improve prognosis of patients vulnerable to a cytokine storm. Our ChaC-MS dissection of G9a-associated pathways revealed a novel mechanism of inhibiting G9a for COVID-19 therapy. By ET-phenotypic interactions with primary components of the SARS-CoV-2 upregulated translation machinery, the enzymatic activity of G9a affects multiple inter-promotional pathways associated with COVID-19-characteristic hyperinflammation, lymphopenia, and virus replication. Accordingly, global translatome profiling of G9a inhibitor-treated ET macrophages identified a profile of G9a-dependent protein overexpression similar to the systemic cytokine profiles observed in COVID-19 patients. Specifically, inhibition of G9a enzymatic activity reduced the expression of eleven ARDS- or sepsis-related proteins: SPP1, CCL2, IL1RN, CXCL2, SQSTM1, ANPEP, PLAU, PELI1, PROCR, DST, and FABP4. These proteins had higher abundances in severe versus mild patients, or mild patients compared with healthy individuals. Thus, unlike most anti-inflammatory therapies that target only one cytokine/chemokine at a time, G9a-targeted therapy can suppress the systemic hyperinflammatory response by simultaneous inhibition of multiple components of a COVID-19 cytokine storm. In parallel, G9a depletion can also restore T cell function and reverse lymphopenia in COVID-19 patients. Because ChaC-MS identified Rps14 and SF3B1, the anti-SARS-CoV-2 targets, as ET-phenotypic G9a interactors, G9a-targeted therapy can be combined with Rps14- or SF3B1-inhibition antiviral therapy to improve the efficacy of single target therapy. Also, because an Ezh2 inhibitor showed inhibitory effects similar to the effects of a G9a inhibitor, the Ezh2 inhibitors with proven safety for cancer therapy can be repurposed for COVID-19 therapy.

### Example 12

### Results of In Vivo Effects of G9a Inhibition on the Peripheral Blood Mononuclear Cells (PBMCs) from COVID-19 Patients

The effect of G9a inhibition on coronavirus-related proteins in clinical setting was also investigated: Ex vivo cultures of UNC0642 treated (0 h, 4 h, 8 h, 12 h and 24 h) PBMCs from a COVID-19 patient were TMT labelled and subjected to mass spectrometry analysis to assess changes in global expression. Of the 5566 proteins quantified in total, 292 proteins were differentially expressed following UNC0642 treatment (ANOVA, P value<0.05; 141 upregulated, 151 downregulated) with turnover of nearly half of these proteins (152 out of 292: 78 upregulated, 74 downregulated) being G9a dependent in ET macrophages (FIG. 4D-4F), meaning they are likely regulated at post-transcriptional/translational level by the G9a complex. Furthermore, several of these G9a-translated proteins were identified as ET-specific G9a interactors, nonhistone G9a substrates or G9a/METTL3-dependent m6A targets, which supported the translation regulatory function of G9a in COVID-19 pathogenesis. Overall, dysregulated proteins were involved in translation (EIF2, mTOR, eIF4 and p70S6K, tRNA charging), immune/viral response (coronavirus pathogenesis, interleukin, CD40, T-cell, endocytosis, phagocytosis), cellular metabolism (nucleotide, fatty-acid, amino-acids) and DDR signaling (NHEJ, BER); all pathways that have consistently been top hits in studies investigating host factors necessary for replication or infection of SARS-CoV-1/2, MFRS and other coronaviruses. Indeed, UNC0642 treatment of patient derived PBMCs led to differential expression of 8 host factors that are necessary for SARS-CoV-2 pathogenesis as identified by siRNA and genome-wide CRISPR screens, 129 host proteins that interact with various SARS-CoV-1/2 and MERS encoded proteins, 2 host interactors of SARS-CoV-2 viral RNA [20], and one protein that predisposes patients to severe COVID-19 infection as characterized by a genome-wide association study. In short, UNC0642 mediated inhibition of G9a in COVID-19 patient derived PBMCs led to globally altered expression of (1) host interactors of SARS-CoV-2 encoded proteins and SARS-CoV-2 viral RNA, (2) host factors required for efficient SARS-CoV-2 infection/replication and (3) the critical pathways involved in coronavirus pathogenesis.

### Discussion of Examples 1-12

The ability to develop targeted therapies to minimize mortality of severe patients depends on a detailed understanding of SARS-CoV-2-dysregulated chronic inflammatory pathways that determine COVID-19 severity. Our results from chemoproteomics, MeRIP-seq, translatome proteomics, and molecular/cell biology revealed a novel mechanism of inhibitor action on COVID-19. Specifically, via ET-phenotypic G9a-interacting translation machinery, SARS-CoV-2 may evolve a G9a-associated mechanism of gene-specific translation activation to modulate host response, evade the host immune system, and promote viral replication and infection. In endotoxin-tolerant macrophages that share similar immunopathologic characteristics with SARS-CoV-2 dysregulated macrophages, the G9a-associated pathways that cause individuals with pre-existing chronic inflammatory diseases to be highly susceptible to secondary infection by SARS-CoV-2 were dissected. Specifically, a widespread translational function of constitutively active G9a in the impairment/depletion of T cell function and the production of organ-damaging cytokine storm was discovered. Importantly, combined results from COVID-19 patients with pre-existing conditions, genome-wide CRISPR screening, and CoV-host interactome mapping validated our mechanistic findings in ET and identified numerous G9a interactors or G9a-translated proteins in the interconnected networks associated, respectively, with the host response to SARS-CoV-2 infection or host-virus interactions. Compared with current trials focused on either antiviral or anti-inflammatory therapy, this single-target inhibition of G9a-associated pathways was responsible for multifaceted, systematic effects, namely, restoration of T cell function to overcome lymphopenia, mitigation of hyperinflammation, and suppression of viral replication. Our studies have paved new roads that combine immunomodulatory and antiviral therapies with more effective COVID-19 therapies with minimal side effects. Importantly, because both G9a and its complex components were overexpressed in infected host cells by other CoV strains (e.g., SARS-CoV-1 and MERS-CoV), our G9a-targeted therapy is refractory to complications induced by emerging antiviral-resistant mutants of SARS-CoV-2, or any virus, that hijacks host responses.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

It will be understood that various details of the presently disclosed subject matter may be changed without departing from the scope of the presently disclosed subject matter. Furthermore, the foregoing description is for the purpose of illustration only, and not for the purpose of limitation.
1. Paranjpe, I. et al. Clinical Characteristics of Hospitalized Covid-19 Patients in New York City. medRxiv (2020).
2. Menon, R. et al. SARS-CoV-2 receptor networks in diabetic kidney disease, BK-Virus nephropathy and COVID-19 associated acute kidney injury. medRxiv (2020).
3. Mehta, P. et al. COVID-19: consider cytokine storm syndromes and immunosuppression. Lancet 395, 1033-1034 (2020).
4. Tay, M. Z., Poh, C. M., Renia, L., MacAry, P. A. & Ng, L. F. P. The trinity of COVID-19: immunity, inflammation and intervention. Nat Rev Immunol 20, 363-374 (2020).
5. Diao, B. et al. Reduction and Functional Exhaustion of T Cells in Patients With Coronavirus Disease 2019 (COVID-19). Front Immunol 11, 827 (2020).
6. Shen, B. et al. Proteomic and Metabolomic Characterization of COVID-19 Patient Sera. Cell (2020).
7. Wang, C. et al. Alveolar macrophage dysfunction and cytokine storm in the pathogenesis of two severe COVID-19 patients. EBioMedicine 57, 102833 (2020).
8. Liao, M. et al. Single-cell landscape of bronchoalveolar immune cells in patients with COVID-19. Nat Med 26, 842-844 (2020).
9. Merad, M. & Martin, J. C. Pathological inflammation in patients with COVID-19: a key role for monocytes and macrophages. Nat Rev Immunol 20, 355-362 (2020).
10. Liu, D. et al. Viral sepsis is a complication in patients with Novel Corona Virus Disease (COVID-19). Med Drug Discov 8, 100057 (2020).
11. Liu, D., Cao, S., Zhou, Y. & Xiong, Y. Recent advances in endotoxin tolerance. J Cell Biochem 120, 56-70 (2019).
12. Lopez-Collazo, E. & del Fresno, C. Pathophysiology of endotoxin tolerance: mechanisms and clinical consequences. Crit Care 17, 242 (2013).
13. Wei, J. et al. Genome-wide CRISPR screens reveal host factors critical for SARS-CoV-2 infection. Cell (2020).
14. Liu, C. et al. A chromatin activity-based chemoproteomic approach reveals a transcriptional repressome for gene-specific silencing. Nat Commun 5, 5733 (2014).
15. Ramlall, V. et al. Immune complement and coagulation dysfunction in adverse outcomes of SARS-CoV-2 infection. Nat Med 26, 1609-1615 (2020).
16. Wrobel, J. A. et al. Multi-omic Dissection of Oncogenically Active Epiproteomes Identifies Drivers of Proliferative and Invasive Breast Tumors. iScience 17, 359-378 (2019).
17. Malovannaya, A. et al. Analysis of the human endogenous coregulator complexome. Cell 145, 787-99 (2011).
18. Huttlin, E. L. et al. The BioPlex Network: A Systematic Exploration of the Human Interactome. Cell 162, 425-40 (2015).
19. Bojkova, D. et al. Proteomics of SARS-CoV-2-infected host cells reveals therapy targets. Nature 583, 469-472 (2020).
20. Lin, S., Choe, J., Du, P., Triboulet, R. & Gregory, R. I. The m(6)A Methyltransferase METTL3 Promotes Translation in Human Cancer Cells. Mol Cell 62, 335-345 (2016).
21. Zong, X. et al. Mett13 Deficiency Sustains Long-Chain Fatty Acid Absorption through Suppressing Traf6-Dependent Inflammation Response. J Immunol 202, 567-578 (2019).
22. Lu, N. et al. Curcumin Attenuates Lipopolysaccharide-Induced Hepatic Lipid Metabolism Disorder by Modification of m(6) A RNA Methylation in Piglets. Lipids 53, 53-63 (2018).
23. Feng, Z., Li, Q., Meng, R., Yi, B. & Xu, Q. METTL3 regulates alternative splicing of MyD88 upon the lipopolysaccharide-induced inflammatory response in human dental pulp cells. J Cell Mol Med 22, 2558-2568 (2018).
24. Ankney, J. A., Muneer, A. & Chen, X. Relative and Absolute Quantitation in Mass Spectrometry-Based Proteomics. Annu Rev Anal Chem (Palo Alto Califi 11, 49-77 (2018).
25. Wang, L. et al. Novel RNA-Affinity Proteogenomics Dissects Tumor Heterogeneity for Revealing Personalized Markers in Precision Prognosis of Cancer. Cell Chem Biol 25, 619-633 e5 (2018).
26. Feltcher, M. E. et al. Label-free Quantitative Proteomics Reveals a Role for the Mycobacterium tuberculosis SecA2 Pathway in Exporting Solute Binding Proteins and Mce Transporters to the Cell Wall. Mol Cell Proteomics 14, 1501-16 (2015).
27. Avendano-Ortiz, J. et al. PD-L1 Overexpression During Endotoxin Tolerance Impairs the Adaptive Immune Response in Septic Patients via HIFI alpha. J Infect Dis 217, 393-404 (2018).
28. Pachot, A. et al. Decreased expression of the fractalkine receptor CX3CR1 on circulating monocytes as new feature of sepsis-induced immunosuppression. J Immunol 180, 6421-9 (2008).
29. Li, J. et al. Virus-Host Interactome and Proteomic Survey Reveal Potential Virulence Factors Influencing SARS-CoV-2 Pathogenesis. Med (N Y) (2020).
30. Gordon, D. E. et al. Comparative host-coronavirus protein interaction networks reveal pan-viral disease mechanisms. Science (2020).
31. Zhang, S. et al. Reversing SKI-SMAD4-mediated suppression is essential for TH17 cell differentiation. Nature 551, 105-109 (2017).
32. Arend, K. C., Lenarcic, E. M. & Moorman, N.J. The 5′ Untranslated Region of the Major Immediate Early mRNA Is Necessary for Efficient Human Cytomegalovirus Replication. J Virol 92(2018).
33. Hamy, A. S. et al. BIRC5 (survivin): a pejorative prognostic marker in stage II/III breast cancer with no response to neoadjuvant chemotherapy. Breast Cancer Res Treat 159, 499-511 (2016).
34. Kidokoro, T. et al. CDC20, a potential cancer therapeutic target, is negatively regulated by p53. Oncogene 27, 1562-71 (2008).
35. Wang, Z. et al. Cdc20: a potential novel therapeutic target for cancer treatment. Curr Pharm Des 19, 3210-4 (2013).
36. Gordon, C. A., Gong, X., Ganesh, D. & Brooks, J. D. NUSAP1 promotes invasion and metastasis of prostate cancer. Oncotarget 8, 29935-29950 (2017).
37. Gill, R. M., Gabor, T. V., Couzens, A. L. & Scheid, M. P. The MYC-associated protein CDCA7 is phosphorylated by AKT to regulate MYC-dependent apoptosis and transformation. Mol Cell Biol 33, 498-513 (2013).
38. Hu, R. et al. SKA3 promotes cell proliferation and migration in cervical cancer by activating the PI3K/Akt signaling pathway. Cancer Cell Int 18, 183 (2018).
39. Mahadevappa, R. et al. The prognostic significance of Cdc6 and Cdtl in breast cancer. Sci Rep 7, 985 (2017).
40. Kobayashi, H. et al. Overexpression of denticleless E3 ubiquitin protein ligase homolog (DTL) is related to poor outcome in gastric carcinoma. Oncotarget 6, 36615-24 (2015).
41. Musa, J., Aynaud, M. M., Mirabeau, O., Delattre, O. & Grunewald, T. G. MYBL2 (B-Myb): a central regulator of cell proliferation, cell survival and differentiation involved in tumorigenesis. Cell Death Dis 8, e2895 (2017).
42. Liu, S. et al. METTL3 plays multiple functions in biological processes. Am J Cancer Res 10, 1631-1646 (2020).
43. Vu, L. P. et al. The N(6)-methyladenosine (m(6)A)-forming enzyme METTL3 controls myeloid differentiation of normal hematopoietic and leukemia cells. Nat Med 23, 1369-1376 (2017).
44. Erdogan, O. et al. Proteomic dissection of LPS-inducible, PHF8-dependent secretome reveals novel roles of PHF8 in TLR4-induced acute inflammation and T cell proliferation. Sci Rep 6, 24833 (2016).
45. Wang, T. et al. Flightless I homolog negatively modulates the TLR pathway. J Immunol 176, 1355-62 (2006).
46. Moore, K. E. et al. A general molecular affinity strategy for global detection and proteomic analysis of lysine methylation. Mol Cell 50, 444-56 (2013).
47. Islam, K. et al. Defining efficient enzyme-cofactor pairs for bioorthogonal profiling of protein methylation. Proc Natl Acad Sci USA 110, 16778-83 (2013).
48. Du, Y. et al. SUMOylation of the m6A-RNA methyltransferase METTL3 modulates its function. Nucleic Acids Res 46, 5195-5208 (2018).
49. Wang, L. et al. Non-canonical Bromodomain within DNA-PKcs Promotes DNA Damage Response and Radioresistance through Recognizing an IR-Induced Acetyl-Lysine on H2AX. Chem Biol 22, 849-61 (2015).
50. Meyer, K. D. et al. 5′ UTR m(6)A Promotes Cap-Independent Translation. Cell 163, 999-1010 (2015).
51. Zhu, H., Pan, S., Gu, S., Bradbury, E. M. & Chen, X. Amino acid residue specific stable isotope labeling for quantitative proteomics. Rapid Commun Mass Spectrom 16, 2115-23 (2002).
52. Schwanhausser, B., Gossen, M., Dittmar, G. & Selbach, M. Global analysis of cellular protein translation by pulsed SILAC. Proteomics 9, 205-9 (2009).
53. Zheng, Y. et al. Inhibition of EHMT1/2 rescues synaptic and cognitive functions for Alzheimer's disease. Brain 142, 787-807 (2019).
54. Kim, Y. et al. Targeting the histone methyltransferase G9a activates imprinted genes and improves survival of a mouse model of Prader-Willi syndrome. Nat Med 23, 213-222 (2017).
55. Islam, K. et al. Bioorthogonal profiling of protein methylation using azido derivative of S-adenosyl-L-methionine. Journal of the American Chemical Society 134, 5909-5915 (2012).
56. Islam, K. et al. Defining efficient enzyme-cofactor pairs for bioorthogonal profiling of protein methylation. Proceedings of the National Academy of Sciences 110, 16778-16783 (2013).
57. Chae, Y.-C. et al. FOXO1 degradation via G9a-mediated methylation promotes cell proliferation in colon cancer. Nucleic acids research 47, 1692-1705 (2019).
58. Arunachalam, P. S. et al. Systems biological assessment of immunity to mild versus severe COVID-19 infection in humans. Science 369, 1210-1220 (2020).
59. Carvelli, J. et al. Association of COVID-19 inflammation with activation of the C5a-axis. Nature, 1-9 (2020).
60. Konze, K. D. et al. An orally bioavailable chemical probe of the Lysine Methyltransferases EZH2 and EZH1. ACS Chem Biol 8, 1324-34 (2013).
61. Mathew, D. et al. Deep immune profiling of COVID-19 patients reveals distinct immunotypes with therapeutic implications. Science (2020).
62. Foster, S. L., Hargreaves, D. C. & Medzhitov, R. Gene-specific control of inflammation by TLR-induced chromatin modifications. Nature 447, 972-8 (2007).
63. Bouhaddou, M. et al. The Global Phosphorylation Landscape of SARS-CoV-2 Infection. Cell 182, 685-712 e19 (2020).
64. Miller, K. et al. Coronavirus interactions with the cellular autophagy machinery. Autophagy, 1-9 (2020).
65. Nie, X. et al. Multi-organ Proteomic Landscape of COVID-19 Autopsies. medRxiv, 2020.08.16.20176065 (2020).
66. Carvelli, J. et al. Association of COVID-19 inflammation with activation of the C5a-axis. Nature (2020).
67. Parackova, Z. et al. Disharmonic Inflammatory Signatures in COVID-19: Augmented Neutrophils' but Impaired Monocytes' and Dendritic Cells' Responsiveness. Cells 9, 2206 (2020).
68. Wiersinga, W. J., Rhodes, A., Cheng, A. C., Peacock, S. J. & Prescott, H. C. Pathophysiology, Transmission, Diagnosis, and Treatment of Coronavirus Disease 2019 (COVID-19): A Review. JAMA (2020).
69. Bian, C., Chen, Q. & Yu, X. Correction: The zinc finger proteins ZNF644 and WIZ regulate the G9a/GLP complex for gene repression. eLife 4, e08168 (2015).
70. Zecha, J. et al. TMT labeling for the masses: a robust and cost-efficient, in-solution labeling approach. Molecular & cellular proteomics 18, 1468-1478 (2019).
71. Weng, Y.-L. et al. Epitranscriptomic m6A regulation of axon regeneration in the adult mammalian nervous system. Neuron 97, 313-325. e6 (2018).
72. Li, H. et al. The sequence alignment/map format and SAMtools. Bioinfonnatics 25, 2078-2079 (2009).
73. Boisvert, F.-M. et al. A quantitative spatial proteomics analysis of proteome turnover in human cells. Molecular & Cellular Proteomics 11(2012).
74. Welle, K. A. et al. Time-resolved analysis of proteome dynamics by tandem mass tags and stable isotope labeling in cell culture (TMT-SILAC) hyperplexing. Molecular & Cellular Proteomics 15, 3551-3563 (2016).
75. Jovanovic, M. et al. Dynamic profiling of the protein life cycle in response to pathogens. Science 347, 1259038 (2015).
76. Ly, T. et al. Proteome-wide analysis of protein abundance and turnover remodelling during oncogenic transformation of human breast epithelial cells. Wellcome open research 3, 51 (2018).

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of treating a subject suffering from symptoms related to a coronavirus infection and/or COVID-19, the method comprising administering to the subject a therapeutically effective amount of an inhibitor of G9a, an inhibitor of Ezh2, and/or combinations thereof.

2. The method of claim 1, wherein the subject is suffering from COVID-19.

3. The method of any of claims 1 to 2, wherein the subject is suffering from SARS-CoV-2 pathologic pathways related to a host response and viral replication from the coronavirus infection and/or COVID-19.

4. The method of any of claims 1 to 2, wherein the subject is suffering from a hyperinflammatory response mediated by SARS-CoV-2-dysregulated macrophage activation resulting in a cytokine storm.

5. The method of claim 1, wherein the G9a inhibitor comprises a small molecule capable of reducing and/or inhibiting translational regulatory processes associated with G9a.

6. The method of claim 1, wherein the G9a inhibitor comprises UNC0642, wherein the EZH2 inhibitor comprises UNC1999.

7. The method of claim 1, wherein the subject is co-administered both the inhibitor of G9a and the inhibitor of Ezh2.

8. The method of claim 1, wherein the administration of the inhibitor of G9a and/or the inhibitor of Ezh2 reduces and/or inhibits coronavirus replication in the subject.

9. The method of claim 1, wherein the administration of the inhibitor of G9a and/or the inhibitor of Ezh2 restores T cell function to overcome lymphopenia, mitigates hyperinflammation, and/or suppresses of viral replication in the subject.

10. The method of claim 1, wherein the administration of the inhibitor of G9a and/or the inhibitor of Ezh2 suppresses a systemic hyperinflammatory response in the subject by simultaneously inhibiting multiple components of a COVID-19 cytokine storm, wherein the components of the COVID-19 cytokine storm that are inhibited are ARDS-related proteins and/or sepsis-related proteins, optionally wherein the ARDS-related proteins and/or sepsis-related proteins are selected from the group consisting of SPP1, CCL2, IL1RN, CXCL2, SQSTM1, ANPEP, PLAU, PELI1, PROCR, DST, and FABP4.

11. The method of claim 1, wherein the inhibitor of G9a or Ezh2 is administered to the subject in a pharmaceutically acceptable formulation or carrier.

12. The method of claim 1, wherein the subject is a human subject.

13. A method of blocking G9a translational regulation of hyperinflammation in a subject, the method comprising administering to the subject a therapeutically effective amount of an inhibitor of G9a, wherein G9a translational regulation of inflammation in the subject is blocked or substantially reduced.

14. The method of claim 13, wherein the subject is suffering from an infection or other condition causing chronic or acute inflammation.

15. The method of claim 13, wherein the subject is suffering from coronavirus viral infection and/or COVID-19.

16. The method of claim 13, wherein the G9a inhibitor comprises a small molecule capable of reducing and/or inhibiting translational regulatory processes associated with G9a.

17. The method of claim 13, wherein the G9a inhibitor comprises UNC0642, wherein the EZH2 inhibitor comprises UNC1999.

18. The method of claim 13, wherein the administration of the inhibitor of G9a suppresses a systemic hyperinflammatory response in the subject by inhibiting ARDS-related proteins and/or sepsis-related proteins, optionally wherein the ARDS-related proteins and/or sepsis-related proteins are selected from the group consisting of SPP1, CCL2, IL1RN, CXCL2, SQSTM1, ANPEP, PLAU, PELI1, PROCR, DST, and FABP4.

19. The method of claim 13, wherein the administration of the inhibitor of G9a blocks METTL3-mediated translational regulation of chronic inflammation in the subject.

20. The method of claim 13, wherein the inhibitor of G9a is administered to the subject in a pharmaceutically acceptable formulation or carrier.

21. The method of claim 13, wherein the subject is a human subject.

22. A method of identifying a compound to treat and/or prevent chronic and/or hyperinflammation in a subject, the method comprising identifying a compound that blocks G9a translational regulation of inflammation in the subject.

23. The method of claim 22, wherein the compound comprises an inhibitor of G9a, optionally a small molecule inhibitor.

24. Use of a composition for treating a subject suffering from symptoms related to a coronavirus infection, comprising administering to the subject the composition comprising a therapeutically effective amount of an inhibitor of G9a, an inhibitor of Ezh2, and/or combinations thereof.

25. The use of claim 24, wherein the subject is suffering from a coronavirus infection and/or COVID-19.

26. The use of any of claims 24 to 25, wherein the subject is suffering from SARS-CoV-2 pathologic pathways related to a host response and viral replication from the coronavirus infection or COVID-19.

27. The use of any of claims 24 to 25, wherein the subject is suffering from a hyperinflammatory response mediated by SARS-CoV-2-dysregulated macrophage activation resulting in a cytokine storm.

28. The use of claim 24, wherein the G9a inhibitor comprises a small molecule capable of reducing and/or inhibiting translational regulatory processes associated with G9a.

29. The use of claim 24, wherein the G9a inhibitor comprises UNC0642, wherein the EZH2 inhibitor comprises UNC1999.

30. The use of claim 24, wherein the subject is co-administered both the inhibitor of G9a and the inhibitor of Ezh2.

31. The use of claim 24, wherein the administration of the inhibitor of G9a and/or the inhibitor of Ezh2 reduces and/or inhibits coronavirus replication and/or infection in the subject.

32. The use of claim 24, wherein the administration of the inhibitor of G9a and/or the inhibitor of Ezh2 restores T cell function to overcome lymphopenia, mitigates hyperinflammation, and/or suppresses of viral replication in the subject.

33. The use of claim 24, wherein the subject is a human subject.


SEQUENCE LISTING (3 sequences)

SEQ ID NO: 1 - DNA, 28 bp, Artificial Sequence
  Artificially synthesized primer nucleotide
  misc_feature (28)..(28): n is a, c, g, t or u
aagcagtggtatcaacgcagagtactvn

SEQ ID NO: 2 - DNA, 33 bp, Artificial Sequence
  Artificially synthesized primer nucleotide
aagcagtggtatcaacgcagagtacatrgrgrg

SEQ ID NO: 3 - DNA, 23 bp, Artificial Sequence
  Artificially synthesized primer nucleotide
aagcagtggtatcaacgcagagt